(19) United States
(12) Patent Application Publication
(10) Pub. No.: US 2007/0264623 A1
(43) Pub. Date: Nov. 15, 2007

(54) Nanosensors

(75) Inventors: Wayne Wang, Cambridge, MA (US); Chuo Chen, Dallas, TX (US); Keng-Hui Lin, Cambridge, MA (US); Ying Fang, Cambridge, MA (US); Charles M. Lieber, Lexington, MA (US)

Correspondence Address: WOLF GREENFIELD & SACKS, P.C., 600 ATLANTIC AVENUE, BOSTON, MA, 02210-2206, US

(73) Assignee: PRESIDENT AND FELLOWS OF HARVARD COLLEGE, Cambridge, MA (US)

(21) Appl. No.: 11/629,722

(22) PCT Filed: Jun. 15, 2005
(86) PCT No.: PCT/US05/20974

§ 371 Date: Jul. 12, 2007

Related U.S. Application Data
(60) Provisional application No. 60/579,996, filed on Jun. 15, 2004.

(51) Int. Cl. C12Q 1/00; C12M 1/40; G01N 27/00; G01N 33/53

(52) U.S. Cl. 435/4; 422/68.1; 422/82.01; 422/82.05; 435/287.1; 436/501

(57) ABSTRACT

The present invention generally relates to nanoscale wires for use in determining analytes suspected to be present in a sample, especially in connection with determining information about a sample containing, or suspected of containing, two or more analytes. For example, the invention can involve a competitive, uncompetitive, or non-competitive binding assay including a nanoscale wire to a sample containing a species able to interact with the retain entity to produce a product, where the sample also contains or is suspected of containing a second species able to interact with the reaction entity to prevent production of the product resulting from interaction of the first species and the reaction entity. Based upon determination of production of the product, determination of the second species in the sample can be made. In one set of embodiments, nanoscale wires can be used that have been functionalized at their surface, and/or in close proximity to their surface, for example, by immobilizing a protein or an enzyme relative to the nanoscale wire. Functionalization may permit interaction of the nanoscale wire with various analytes, and such interaction may induce a determinable change in a property of the nanoscale wire. Determination of two or more analytes, o one analyte and the suspected presence of another analyte can involve, for example, binding species to a protein or an enzyme immobilized relative to the nanoscale wire. Other aspects of the invention include assays, sensors, detectors, and/or other devices that include functionalized nanoscale wires, methods of making and/or using functionalized nanoscale wires (for example, in drug screening or high throughput screening) and the like.

### FIELD OF INVENTION

The present invention relates generally to nanoscale devices and methods, and more particularly to nanoscale wires for use in binding assays to determine analytes suspected to be present in a sample.

### BACKGROUND OF THE INVENTION

Interest in nanotechnology, in particular sub-microelectronic technologies such as semiconductor quantum dots and nanowires, has been motivated by the challenges of chemistry and physics at the nanoscale, and by the prospect of utilizing these structures in electronic and related devices. Nanoscopic articles might be well-suited for transport of charge carriers and excitons (e.g. electrons, electron pairs, etc.) and thus may be useful as building blocks in nanoscale electronics applications. Nanowires are ideally suited for efficient transport of charge carriers and excitons, and thus are expected to be critical building blocks for nanoscale electronics and optoelectronics.

Nanowires having selectively functionalized surfaces have been described in U.S. patent application Ser. No. 10/020,004, entitled “Nanosensors,” filed Dec. 11, 2001, published as Publication No. 2002/0117659 on Aug. 29, 2002, and as corresponding International Patent Publication WO02/48701, published Jun. 20, 2002. In described, functionalization of the nanowire permits interaction of the functionalized nanowire with various entities, such as molecular entities, and the interaction induces a change in a property of the functionalized nanowire, which provides a mechanism for a nanoscale sensor device for detecting the presence or absence of an analyte suspected to be present in a sample.

### SUMMARY OF THE INVENTION

The present invention generally relates to nanoscale wires for use in binding assays to determine analytes suspected to be present in a sample. The subject matter of the present invention involves, in some cases, interrelated products, alternative solutions to a particular problem, and/or a plurality of different uses of one or more systems and/or articles.

One aspect of the invention provides a system. The system, in one set of embodiments, includes a sample exposure region comprising a reaction entity associated with a nanoscale wire, and a first species and a second species different from the first species, each within the sample exposure region. Each of the first and second species may be able to interact with the reaction entity or to affect interaction of the reaction entity with the other species.

Another aspect of the invention provides a method. The method, in one set of embodiments, includes an act of exposing a reaction entity associated with a nanoscale wire to a sample containing a first species and containing or suspected of containing a second species different from the first species. Each species may be able to interact with the reaction entity and/or able to affect the interaction of the other species with the reaction entity. In another set of embodiments, the method may include acts of exposing a nanoscale wire to an analyte, and determining a binding constant between the analyte and the nanoscale wire.

In another aspect, the present invention is directed to a method of making one or more of the embodiments described herein. In yet another aspect, the present invention is directed to a method of using one or more of the embodiments described herein. In still another aspect, the present invention is directed to a method of promoting one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more applications incorporated by reference include conflicting and/or inconsistent disclosure with respect to each other, then the later-filed application shall control.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIGS. 1A-1B schematically illustrates a nanoscale detector device having a binding agent, according to one embodiment of the invention;

FIGS. 2A-2B schematically illustrate certain nanoscale detector devices that can be used in connection with the invention;

FIGS. 3A-3D illustrate an embodiment of a nanoscale detector, as used in a field effect transistor, that can be used in connection with;

FIGS. 4A-4C illustrate certain small molecule-protein interactions;

FIGS. 5A-5B illustrate the determination of ATP binding, according to one embodiment of the invention;

FIGS. 6A-6B illustrate determination of the inhibition of ATP binding, according to another embodiment of the invention; and

FIGS. 7A-7C illustrate the screening of small molecule inhibitors, in accordance with another embodiment of the invention.

### DETAILED DESCRIPTION

The present invention relates to nanoscale wires for use in determining analytes suspected of being present in a sample, especially in connection with determining information about a sample containing, or suspected of containing, two or more analytes, or determining the interaction between chemical or biological species in the presence of other species that can affect this interaction. It is a feature of the invention that, while prior studies have demonstrated the ability to detect the quantity and/or presence of an analyte in a sample to which a nanowire is exposed, the present invention provides the ability to determine not only whether a species is in proximity of a nanoscale wire, but which of two species, placed in proximity of the nanoscale wire, is involved in a particular binding event. In one set of embodiments, the nanoscale wire can be used to distinguish which of two species have bound to a location proximate the wire. In another set of embodiments the wire can be used to determine whether a particular binding event has occurred, allowing determination about a different binding event.

For example, the invention can involve a competitive, uncompetitive, or non-competitive binding assay including a nanoscale wire, which involves exposing a reaction entity associated with the nanoscale wire to a sample containing a species able to interact with the reaction entity to produce a product, where the sample also contains or is suspected of containing a second species able to interact with the reaction entity to prevent production of the product resulting from interaction of the first species and the reaction entity. Based upon determination of production of the product, determination of the second species in the sample can be made.

In one set of embodiments, nanoscale wires can be used that have been functionalized at their surface, and/or in close proximity to their surface, for example, by immobilizing a protein or an enzyme relative to the nanoscale wire. Functionalization (for example, with a reaction entity), either uniformly or non-uniformly, may permit interaction of the nanoscale wire with various analytes, and such interaction may induce a determinable change in a property of the nanoscale wire. Determination of two or more analytes, or one analyte and the suspected presence of another analyte, as discussed above, can involve, for example, binding a species to a protein or an enzyme immobilized relative to the nanoscale wire. In some cases, the analytes may competitively, uncompetitively, or noncompetitively interact with the functionalized nanoscale wire. The surface of the nanowires may also be selectively functionalized in some instances. Other aspects of the invention include assays, sensors, detectors, and/or other devices that include functionalized nanoscale wires, methods of making and/or using functionalized nanoscale wires (for example, in drug screening or high throughput screening), and the like.

Definitions

The following definitions will aid in the understanding of the invention. Certain devices of the invention may include wires or other components of scale commensurate with nanometer-scale wires, which includes nanotubes and nanowires. In some embodiments, however, the invention comprises articles that may be greater than nanometer size (e. g., micrometer-sized). As used herein, “nanoscopic-scale,” “nanoscopic,” “nanometer-scale,” “nanoscale,” the “nano-” prefix (for example, as in “nanostructured”), and the like generally refers to elements or articles having widths or diameters of less than about 1 micron, and less than about 100 nm in some cases. In all embodiments, specified widths can be a smallest width (i.e. a width as specified where, at that location, the article can have a larger width in a different dimension), or a largest width (i.e. where, at that location, the article has a width that is no wider than as specified, but can have a length that is greater).

As used herein, a “wire” generally refers to any material having a conductivity of or of similar magnitude to any semiconductor or any metal, and in some embodiments may be used to connect two electronic components such that they are in electronic communication with each other. For example, the terms “electrically conductive” or a “conductor” or an “electrical conductor” when used with reference to a “conducting” wire or a nanoscale wire, refers to the ability of that wire to pass charge. Typically, an electrically conductive nanoscale wire will have a resistivity comparable to that of metal or semiconductor materials, and in some cases, the electrically conductive nanoscale wire may have lower resistivities, for example, resistivities of less than about 100 microOhm cm (μΩcm). In some cases, the electrically conductive nanoscale wire will have a resistivity lower than about 10⁻³ ohm meters, lower than about 10⁻⁴ ohm meters, or lower than about 10⁻⁶ ohm meters or 10⁻⁷ ohm meters.

A “semiconductor,” as used herein, is given its ordinary meaning in the art, i.e., an element having semiconductive or semi-metallic properties (i.e., between metallic and non-metallic properties). An example of a semiconductor is silicon. Other non-limiting examples include gallium, germanium, diamond (carbon), tin, selenium, tellurium, boron, or phosphorous.

A “nanoscopic wire” (also known herein as a “nanoscopic-scale wire” or “nanoscale wire”) generally is a wire, that at any point along its length, has at least one cross-sectional dimension and, in some embodiments, two orthogonal cross-sectional dimensions less than 1 micron, less than about 500 nm, less than about 200 nm, less than about 150 nm, less than about 100 nm, less than about 70, less than about 50 nm, less than about 20 nm, less than about 100 nm, or less than about 5 nm. In other embodiments, the cross-sectional dimension can be less than 2 nm or 1 nm. In one set of embodiments, the nanoscale wire has at least one cross-sectional dimension ranging from 0.5 nm to 100 nm or 200 nm. In some cases, the nanoscale wire is electrically conductive. Where nanoscale wires are described having, for example, a core and an outer region, the above dimensions generally relate to those of the core. The cross-section of a nanoscopic wire may be of any arbitrary shape, including, but not limited to, circular, square, rectangular, annular, polygonal, or elliptical, and may be a regular or an irregular shape. The nanoscale wire may be solid or hollow. A non-limiting list of examples of materials from which nanowires of the invention can be made appears below. Any nanoscale wire can be used in any of the embodiments described herein, including carbon nanotubes, molecular wires (i.e., wires formed of a single molecule), nanorods, nanowires, nanowhiskers, organic or inorganic conductive or semiconducting polymers, and the like, unless otherwise specified. Other conductive or semiconducting elements that may not be molecular wires, but are of various small nanoscopic-scale dimensions, can also be used in some instances, e.g. inorganic structures such as main group and metal atom-based wire-like silicon, transition metal-containing wires, gallium arsenide, gallium nitride, indium phosphide, germanium, cadmium selenide, etc. A wide variety of these and other nanoscale wires can be grown on and/or applied to surfaces in patterns useful for electronic devices in a manner similar to techniques described herein involving the specific nanoscale wires used as examples, without undue experimentation. The nanoscale wires, in some cases, may be formed having dimensions of at least about 1 micron, at least about 3 microns, at least about 5 microns, or at least about 10 microns or about 20 microns in length, and can be less than about 100 nm, less than about 80 nm, less than about 60 nm, less than about 40 run, less than about 20 nm, less than about 10 nm, or less than about 5 nm in thickness (height and width). The nanoscale wires may have an aspect ratio (length to thickness) of greater than about 2:1, greater than about 3:1, greater than about 4:1, greater than about 5:1, greater than about 10:1, greater than about 25:1, greater than about 50:1, greater than about 75:1, greater than about 100:1, greater than about 150:1, greater than about 250:1, greater than about 500:1, greater than about 750:1, or greater than about 1000:1 or more in some cases.

A “nanowire” (e. g. comprising silicon and/or another semiconductor material) is a nanoscopic wire that is typically a solid wire, and may be elongated in some cases. Preferably, a nanowire (which is abbreviated herein as “NW”) is an elongated semiconductor, i.e., a nanoscale semiconductor. A “non-nanotube nanowire” is any nanowire that is not a nanotube. In one set of embodiments of the invention, a non-nanotube nanowire having an unmodified surface (not including an auxiliary reaction entity not inherent in the nanotube in the environment in which it is positioned) is used in any arrangement of the invention described herein in which a nanowire or nanotube can be used.

As used herein, a “nanotube” (e.g. a carbon nanotube) is a nanoscopic wire that is hollow, or that has a hollowed-out core, including those nanotubes known to those of ordinary skill in the art. “Nanotube” is abbreviated herein as “NT.” Nanotubes are used as one example of small wires for use in the invention and, in certain embodiments, devices of the invention include wires of scale commensurate with nanotubes.

As used herein, an “elongated” article (e.g. a semiconductor or a section thereof) is an article for which, at any point along the longitudinal axis of the article, the ratio of the length of the article to the largest width at that point is greater than 2:1.

A “width” of an article, as used herein, is the distance of a straight line from a point on a perimeter of the article, through the center of the article, to another point on the perimeter of the article. As used herein, a “width” or a “cross-sectional dimension” at a point along a longitudinal axis of an article is the distance along a straight line that passes through the center of a cross-section of the article at that point and connects two points on the perimeter of the cross-section. The “cross-section” at a point along the longitudinal axis of an article is a plane at that point that crosses the article and is orthogonal to the longitudinal axis of the article. The “longitudinal axis” of an article is the axis along the largest dimension of the article. Similarly, a “longitudinal section” of an article is a portion of the article along the longitudinal axis of the article that can have any length greater than zero and less than or equal to the length of the article. Additionally, the “length” of an elongated article is a distance along the longitudinal axis from end to end of the article.

As used herein, a “cylindrical” article is an article having an exterior shaped like a cylinder, but does not define or reflect any properties regarding the interior of the article. In other words, a cylindrical article may have a solid interior, may have a hollowed-out interior, etc. Generally, a cross-section of a cylindrical article appears to be circular or approximately circular, but other cross-sectional shapes are also possible, such as a hexagonal shape. The cross-section may have any arbitrary shape, including, but not limited to, square, rectangular, or elliptical. Regular and irregular shapes are also included.

As used herein, an “array” of articles (e.g., nanoscopic wires) comprises a plurality of the articles, for example, a series of aligned nanoscale wires, which may or may not be in contact with each other. As used herein, a “crossed array” or a “crossbar array” is an array where at least one of the articles contacts either another of the articles or a signal node (e.g., an electrode).

The invention provides, in certain embodiments, a nanoscale wire or wires forming part of a system constructed and arranged to determine an analyte in a sample to which the nanoscale wire(s) is exposed. “Determine,” in this context, generally refers to the analysis of a species, for example, quantitatively or qualitatively, and/or the detection of the presence or absence of the species. “Determining” may also refer to the analysis of an interaction between two or more species, for example, quantitatively or qualitatively, and/or by detecting the presence or absence of the interaction, e.g. determination of the binding between two species. As an example, an analyte may cause a determinable change in an electrical property of a nanoscale wire (e.g., electrical conductivity), a change in an optical property of the nanoscale wire, etc. Examples of determination techniques include, but are not limited to, piezoelectric measurement, electrochemical measurement, electromagnetic measurement, photodetection, mechanical measurement, acoustic measurement, gravimetric measurement and the like. “Determining” also means detecting or quantifying interaction between species.

The term “electrically coupled” or “electrocoupling,” when used with reference to a nanoscale wire and an analyte, or other moiety such as a reaction entity, refers to an association between any of the analyte, other moiety, and the nanoscale wire such that electrons can move from one to the other, or in which a change in an electrical characteristic of one can be determined by the other. This can include electron flow between these entities, or a change in a state of charge, oxidation, or the like that can be determined by the nanoscale wire. As examples, electrical coupling can include direct covalent linkage between the analyte or other moiety and the nanoscale wire, indirect covalent coupling (e.g. via a linker), direct or indirect ionic bonding between the analyte (or other moiety) and the nanoscale wire, or other bonding (e.g. hydrophobic bonding). In some cases, no actual bonding may be required and the analyte or other moiety may simply be contacted with the nanoscale wire surface. There also need not necessarily be any contact between the nanoscale wire and the analyte or other moiety where the nanoscale wire is sufficiently close to the analyte to permit electron tunneling between the analyte and the nanoscale wire.

As used herein, a component that is “immobilized relative to” another component either is fastened to the other component or is indirectly fastened to the other component, e.g., by being fastened to a third component to which the other component also is fastened. For example, a first entity is immobilized relative to a second entity if a species fastened to the surface of the first entity attaches to an entity, and a species on the surface of the second entity attaches to the same entity, where the entity can be a single entity, a complex entity of multiple species, another particle, etc. In certain embodiments, a component that is immobilized relative to another component is immobilized using bonds that are stable, for example, in solution or suspension. In some embodiments, non-specific binding of a component to another component, where the components may easily separate due to solvent or thermal effects, is not preferred.

As used herein, “fastened to or adapted to be fastened to,” as used in the context of a species relative to another species or a species relative to a surface of an article (such as a nanoscale wire), or to a surface of an article relative to another surface, means that the species and/or surfaces are chemically or biochemically linked to or adapted to be linked to, respectively, each other via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a nanoscale wire, a binding species specifically biologically coupled to an antibody which is bound to a protein such as protein A, which is attached to a nanoscale wire, a binding species that forms a part of a molecule, which in turn is specifically biologically bound to a binding partner covalently fastened to a surface of a nanoscale wire, etc. A species also is adapted to be fastened to a surface if a surface carries a particular nucleotide sequence, and the species includes a complementary nucleotide sequence.

“Specifically fastened” or “adapted to be specifically fastened” means a species is chemically or biochemically linked to or adapted to be linked to, respectively, another specimen or to a surface as described above with respect to the definition of “fastened to or adapted to be fastened,” but excluding essentially all non-specific binding. “Covalently fastened” means fastened via essentially nothing other than one or more covalent bonds.

The term “binding” refers to the interaction between a corresponding pair of molecules or surfaces that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones and the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc.

The term “binding partner” refers to a molecule that can undergo binding with a particular molecule. Biological binding partners are examples. For example, Protein A is a binding partner of the biological molecule IgG, and vice versa. Other non-limiting examples include nucleic acid-nucleic acid binding, nucleic acid-protein binding, protein-protein binding, enzyme-substrate binding, receptor-ligand binding, receptor-hormone binding, antibody-antigen binding, etc. Binding partners include specific, semi-specific, and non-specific binding partners as known to those of ordinary skill in the art. For example, Protein A is usually regarded as a “non-specific” or semi-specific binder. The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair the ligand would specifically and/or preferentially select its receptor from a complex mixture of molecules, or vice versa An enzyme would specifically bind to its substrate, a nucleic acid would specifically bind to its complement, an antibody would specifically bind to its antigen. Other examples include nucleic acids that specifically bind (hybridize) to their complement, antibodies specifically bind to their antigen, binding pairs such as those described above, and the like. The binding may be by one or more of a variety of mechanisms including, but not limited to ionic interactions, and/or covalent interactions, and/or hydrophobic interactions, and/or van der Waals interactions, etc.

A “fluid,” as used herein, generally refers to a substance that tends to flow and to conform to the outline of its container. Typically, fluids are materials that are unable to withstand a static shear stress. When a shear stress is applied to a fluid, it experiences a continuing and permanent distortion. Typical fluids include liquids and gases, but may also include free-flowing solid particles, viscoelastic fluids, and the like.

The term “sample” refers to any cell, tissue, or fluid from a biological source (a “biological sample”), or any other medium, biological or non-biological, that can be evaluated in accordance with the invention. A sample includes, but is not limited to, a biological sample drawn from an organism (e.g. a human, a non-human mammal, an invertebrate, a plant, a fungus, an algae, a bacteria, a virus, etc.), a sample drawn from food designed for human consumption, a sample including food designed for animal consumption such as livestock feed, milk, an organ donation sample, a sample of blood destined for a blood supply, a sample from a water supply, or the like. One example of a sample is a sample drawn from a human or animal to determine the presence or absence of a specific nucleic acid sequence.

A “sample suspected of containing” a particular component means a sample with respect to which the content of the component is unknown. For example, a fluid sample from a human suspected of having a disease, such as a neurodegenerative disease, but not known to have the disease, defines a sample suspected of containing neurodegenerative disease. “Sample” in this context includes naturally-occurring samples, such as physiological samples from humans or other animals, samples from food, livestock feed, etc. Typical samples include tissue biopsies, cells, whole blood, serum or other blood fractions, urine, ocular fluid, saliva, cerebro-spinal fluid, fluid or other samples from tonsils, lymph nodes, needle biopsies, etc.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

As used herein, terms such as “polynucleotide” or “oligonucleotide” or grammatical equivalents generally refer to a polymer of at least two nucleotide bases covalently linked together, which may include, for example, but not limited to, natural nucleosides (e.g., adenosine, thymidine, guanosine, cytidine, uridine, deoxyadenosine, deoxythymidine, deoxyguanosine and deoxycytidine), nucleoside analogs (e.g., 2-aminoadenosine, 2-thiothymidine, inosine, pyrrolopyrimidine, 3-methyladenosine, C5-bromouridine, C5-fluorouridine, C5-iodouridine, C5-propynyluridine, C5-propynylcytidine, C5-methylcytidine, 7-deazaadenosine, 7-deazaguanosine, 8-oxoadenosine, 8-oxoguanosine, O6-methylguanosine, 2-thiocytidine, 2-aminopurine, 2-amino-6-chloropurine, 2,6-diaminopurine, hypoxanthine), chemically or biologically modified bases (e.g., methylated bases), intercalated bases, modified sugars (2′-fluororibose, arabinose, or hexose), modified phosphate moieties (e.g., phosphorothioates or 5′-N-phosphoramidite linkages), and/or other naturally and non-naturally occurring bases substitutable into the polymer, including substituted and unsubstituted aromatic moieties. Other suitable base and/or polymer modifications are well-known to those of skill in the art. Typically, an “oligonucleotide” is a polymer having 20 bases or less, and a “polynucleotide” is a polymer having at least 20 bases. Those of ordinary skill in the art will recognize that these terms are not precisely defined in terms of the number of bases present within the polymer strand.

A “nucleic acid,” as used herein, is given its ordinary meaning as used in the art. Nucleic acids can be single-stranded or double stranded, and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111: 2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp. 169-176). Several nucleic acid analogs are described in Rawls, Chemical & Engineering News, Jun. 2, 1997 page 35. These modifications of the ribose-phosphate backbone may be done to facilitate the addition of additional moieties such as labels, or to increase the stability and half-life of such molecules in physiological environments.

As used herein, an “antibody” refers to a protein or glycoprotein including one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′2, a dimer of Fab which itself is a light chain joined to V_{H}-C_{H}1 by a disulfide bond. The F(ab)′2 may be reduced under mild conditions to break the disulfide linkage in the hinge region thereby converting the (Fab′)2 dimer into an Fab′ monomer. The Fab′ monomer is essentially a Fab with part of the hinge region (see, Paul (1993) Fundamental Immunology, Raven Press, N.Y. for a more detailed description of other antibody fragments). While various antibody fragments are defined in terms of the digestion of an intact antibody, one of skill will appreciate that such fragments may be synthesized de novo either chemically, by utilizing recombinant DNA methodology, or by “phage display” methods (see, e.g., Vaughan et al. (1996) Nature Biotechnology, 14(3): 309-314, and PCT/US96/10287). Preferred antibodies include single chain antibodies, e.g., single chain Fv (scFv) antibodies in which a variable heavy and a variable light chain are joined together (directly or through a peptide linker) to form a continuous polypeptide.

The term “quantum dot” is known to those of ordinary skill in the art, and generally refers to semiconductor or metal nanoparticles that absorb light and quickly re-emit light in a different color depending on the size of the dot. For example, a 2 nanometer quantum dot emits green light, while a 5 nanometer quantum dot emits red light. Cadmium Selenide quantum dot nanocrystals are available from Quantum Dot Corporation of Hayward, Calif.

The following U.S. provisional and utility patent application documents are incorporated herein by reference in their entirety for all purposes, and include additional description of teachings usable with the present invention: Ser. No. 60/142,216, entitled “Molecular Wire-Based Devices and Methods of Their Manufacture,” filed Jul. 2, 1999; Ser. No. 60/226,835, entitled “Semiconductor Nanowires,” filed Aug. 22, 2000; Ser. No. 10/033,369, entitled “Nanoscopic Wire-Based Devices and Arrays,” filed Oct. 24, 2001, published as Publication No 2002/0130353 on Sep. 19, 2002; Ser. No. 60/254,745, entitled “Nanowire and Nanotube Nanosensors,” filed Dec. 11, 2000; Ser. No. 60/292,035, entitled “Nanowire and Nanotube Nanosensors,” filed May 18, 2001; Ser. No. 60/292,121, entitled “Semiconductor Nanowires,” filed May 18, 2001; Ser. No. 60/292,045, entitled “Nanowire Electronic Devices Including Memory and Switching Devices,” filed May 18, 2001; Ser. No. 60/291,896, entitled “Nanowire Devices Including Emissive Elements and Sensors,” filed May 18, 2001; Ser. No. 09/935,776, entitled “Doped Elongated Semiconductors, Growing Such Semiconductors, Devices Including Such Semiconductors, and Fabricating Such Devices,” filed Aug. 22, 2001, published as Publication No. 2002/0130311 on Sep. 19, 2002; Ser. No. 10/020,004, entitled “Nanosensors,” filed Dec. 11, 2001, published as Publication No. 2002/0117659 on Aug. 29, 2002; Ser. No. 60/348,313, entitled “Transistors, Diodes, Logic Gates and Other Devices Assembled from Nanowire Building Blocks,” filed Nov. 9, 2001; Ser. No. 60/354,642, entitled “Nanowire Devices Including Emissive Elements and Sensors,” filed Feb. 6, 2002; Ser. No. 10/152,490, entitled “Nanoscale Wires and Related Devices,” filed May 20, 2002; Ser. No. 10/196,337, entitled “Nanoscale Wires and Related Devices,” filed Jul. 16, 2002, published as Publication No. 2003/0089899 on May 15, 2003; Ser. No. 60/397,121, entitled “Nanowire Coherent Optical Components,” filed Jul. 19, 2002; Ser. No. 10/624,135, entitled “Nanowire Coherent Optical Components,” filed Jul. 21, 2003 Ser. No. 10/734,086, entitled “Nanowire Coherent Optical Components,” filed Dec. 11, 2003; Ser. No. 60/524,301, entitled “Nanoscale Arrays and Related Devices,” filed Nov. 20, 2003; Ser. No. 60/551,634, entitled “Robust Nanostructures,” filed Mar. 8, 2004; and Ser. No. 60/544,800, entitled “Nanostructures Containing Metal-Semiconductor Compounds,” filed Feb. 13, 2004. The following International Patent Publication is incorporated herein by reference in their entirety for all purposes: Application Serial No. PCT/US00/18138, entitled “Nanoscopic Wire-Based Devices, Arrays, and Methods of Their Manufacture,” filed Jun. 30, 2000, published as Publication No. WO 01/03208 on Jan. 11, 2001; Application Serial No. PCT/US01/26298, entitled “Doped Elongated Semiconductors, Growing Such Semiconductors, Devices Including Such Semiconductors, and Fabricating Such Devices,” filed Aug. 22, 2001, published as Publication No. WO 02/17362 on Feb. 28, 2002; Application Serial No. PCT/US01/48230, entitled “Nanosensors,” filed Dec. 11, 2001, published as Publication No. WO 02/48701 on Jun. 20, 2002; Application Serial No. PCT/US02/16133, entitled “Nanoscale Wires and Related Devices,” filed May 20, 2002, published as Publication No. WO 03/005450 on Jan. 16, 2003; Application Serial No. PCT/US03/22061, entitled “Nanoscale Wires and Related Devices,” filed Jul. 16, 2003; and Application Serial No. PCT/US03/11078, entitled “Nanowire Coherent Optical Components,” filed Jul. 21, 2003, published as Publication No. WO 2004/010552 on Jan. 29, 2004.

Embodiments

As noted above, the present invention relates generally to nanoscale wires for use in determining analytes suspected to be present in a sample, especially in connection with determining information about a sample containing, or suspected of containing, two or more analytes, for example in connection with competitive, uncompetitive, or non-competitive binding including drug screening and the like. One aspect of the present invention provides a sensing element comprising a nanoscale wire able to interact with one or more analytes. The nanoscale wire may inherently have an ability to interact with the analytes, and/or the nanoscale wire may have a reaction entity able to interact with the analytes. Nanoscale sensing elements of the invention may be used, for example, to determine pH or metal ions, proteins, nucleic acids (e.g. DNA, RNA, etc.), drugs, sugars, carbohydrates, or other analytes of interest, as further described below. In some cases, the sensing element includes a detector constructed and arranged to be able to determine a change in an property of the nanoscale wire, for example, an electrical change, an electromagnetic change, a change in light emission, a change in stress or shape, etc. In one set of embodiments, at least a portion of the nanoscale wire is addressable by a sample containing, or suspected of containing, the analyte(s). The phrase “addressable by a fluid” is defined as the ability of the fluid to be positioned relative to the nanoscale wire so that the analytes suspected of being in the fluid are able to interact with the nanoscale wire. The fluid may be proximate to or in contact with the nanoscale wire.

In some embodiments, more than one analyte may interact with the nanoscale wire, for example, directly, and/or with a reaction entity associated with the nanoscale wire. Each of the analytes may independently be any of the analytes described herein, for example, proteins, small molecules, peptides, drugs or drug candidates, hormones, vitamins, ligands, sugars, carbohydrates, nucleic acids, etc. In some cases, the two or more analytes may competitively bind to the reaction entity, i.e., the two or more analytes may each be able to bind to the same reaction site on the reaction entity. In other cases, the two or more analytes may noncompetitively bind to the reaction entity, i.e., one analyte may bind to a first reaction site on the reaction entity, and the other analyte may independently bind to a second reaction site on the reaction entity. In still other cases, the two or more analytes may uncompetitively bind to the reaction entity, i.e., the one analytes may bind to a first reaction site on the reaction entity, which alters (enhances or inhibits) the ability of a second analyte to bind to a second reaction site on the reaction entity. “Inhibit”, in this context, can mean to reduce, or to completely eliminate. In one example, a nanoscale wire and/or a reaction entity associated with the nanoscale wire may be exposed to at least a first analyte and a second analyte, and the degree of binding or interaction (e.g., a binding constant) between the analytes and the reaction entity and/or the nanowire (e.g., competitively, noncompetitively, uncompetitively, etc.), may be determined, providing for the measurement of a binding constant between an analyte and an nanoscale wire. One example is in a drug screening technique, as described more fully below.

In one set of embodiments, the nanoscale wire includes, inherently, the ability to determine the analyte. The nanoscale wire, or at least a portion of the nanoscale wire, may be “functionalized,” i.e. the nanoscale wire may comprise one or more surface functional moieties, to which analytes are able to bind and induce a determinable property change in the nanoscale wire. The binding events can be specific or non-specific. In one embodiment, the functional moieties includes one or more simple functional groups, for example, but not limited to, —OH, —CHO, —COOH, —SO₃H, —CN, —NH₂, —SH, —COSH, —COOR, halides, etc. In some cases, a chemical change associated with the nanoscale wire can be used to modulate a property of the nanoscale wire. For example, the presence of the analyte can change an electrical properties of the nanoscale wires, e.g., through electrocoupling with the nanoscale wire.

In another set of embodiments, a reaction entity is associated with the nanoscale wire and is able to interact with the analytes. The reaction entity, as “associated” with the wire, may be positioned in relation to the nanoscale wire (in close proximity or in contact) such that the analyte can be determined by determining a change in a characteristic or property of the nanoscale wire. Interaction of the analyte with the reaction entity may change or modulate a property of the nanoscale wire, for example, through electrocoupling with the reaction entity.

As used herein, the term “reaction entity” refers to any entity that can interact with an analyte in such a manner to cause a detectable change in a property of a nanoscale wire. The reaction entity may enhance the interaction between the nanoscale wire and the analyte, or generate a new chemical species that has a higher affinity to the nanoscale wire, to enrich the analyte around the nanoscale wire, etc. The reaction entity can comprise a binding partner to which the analyte binds. The reaction entity, when a binding partner, can comprise a specific binding partner of the analyte. For example, the reaction entity may be a nucleic acid, an antibody, a sugar, a carbohydrate or a protein. Alternatively, the reaction entity may be a polymer, catalyst, or a quantum dot. A reaction entity that is a catalyst can catalyze a reaction involving the analyte, resulting in a product that causes a determinable change in the nanoscale wire, e.g. via binding to an auxiliary binding partner of the product electrically coupled to the nanoscale wire. Another example of a reaction entity is a reactant that reacts with an analyte, producing a product that can cause a determinable change in the nanoscale wire. In some cases, the reaction entity can comprise a coating on the nanoscale wire, e.g. a coating of a polymer that recognizes molecules in, for instance, a gaseous sample, causing a change in conductivity of the polymer which, in turn, causes a detectable change in the nanoscale wire.

The reaction entity may be positioned relative to the nanoscale wire to cause a detectable change in the nanoscale wire. In some cases, the reaction entity may be positioned within 100 nm of the nanoscale wire, within 50 nm of the nanoscale wire, or within 10 nm of the nanoscale wire. The actual proximity can be determined by those of ordinary skill in the art. Thus, in some cases, the reaction entity is positioned less than 5 nm from the nanoscopic wire. In other cases, the reaction entity is positioned with 4 nm, 3 nm, 2 nm, and 1 nm of the nanoscopic wire. In some cases, the reaction entity may be fastened on the nanoscale wire, for example, through the use of covalent bonds. In other cases, the reaction entity may be immobilized relative to the nanoscale wire, for example, the reaction entity may be attached to the nanoscale wire through a linker.

One example of a reaction entity is a grafted polymer chain with chain length less than the diameter of the nanoscale wire. Examples of suitable polymers include, but are not limited to, polyamide, polyester, polyimide, polyacrylic, and copolymers and blends of these and/or other polymers. Another example of a reaction entity is a surface coating covering the surface of the nanoscale wire, and/or a portion thereof. Non-limiting examples of suitable coating materials include metals, semiconductors, and insulators, which may be a metallic element, an oxide, an sulfide, a nitride, a selenide, a polymer and a polymer gel, as well as combinations of these and/or other materials. Another example of a reaction entity is a biomolecular entity, for example, a member of a binding partner pair. Other non-limiting examples of biomolecular reaction entities include amino acids, proteins, sugars, DNA, antibodies, antigens, and enzymes.

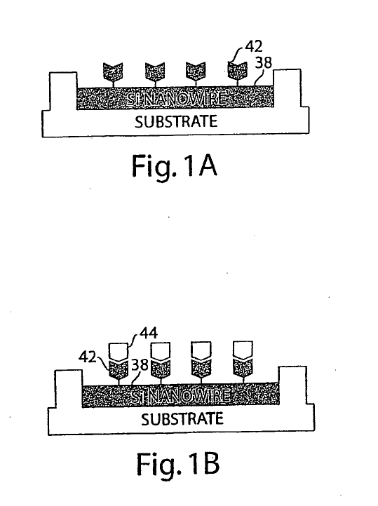

FIG. 1A schematically shows a portion of a nanoscale detector device in which nanoscale wire 38 has been modified with a reactive entity that is a binding partner 42 for detecting analyte 44. FIG. 1B schematically shows a portion of the nanoscale detector device of FIG. 1A, in which the analyte 44 is attached to the specific binding partner 42. Selectively functionalizing the surface of nanowires can be done, for example, by functionalizing the nanoscale wire with a siloxane derivative. For example, a nanoscale wire may be modified after construction of the nanoscale detector device by immersing the device in a solution containing the modifying chemicals to be coated. Alternatively, a micro-fluidic channel may be used to deliver the chemicals to the nanoscale wires. For example, amine groups may be attached by first making the nanoscale detector device hydrophilic by oxygen plasma, or an acid and/or oxidizing agent and the immersing the nanoscale detector device in a solution containing amino silane. By way of example, DNA probes may be attached by first attaching amine groups as described above, and immersing the modified nanoscale detector device in a solution containing bifunctional crosslinkers, if necessary, and immersing the modified nanoscale detector device in a solution containing the DNA probe. The process may be accelerated and promoted by applying a bias voltage to the nanoscale wire, the bias voltage can be either positive or negative depending on the nature of reaction species, for example, a positive bias voltage will help to bring negatively charged DNA probe species close to the nanoscale wire surface and increase its reaction chance with the surface amino groups.

Also provided, according to another set of embodiments, is a sensing element comprising a nanoscale wire and a detector constructed and arranged to determine a change in a property of the nanoscale wire. Where a detector is present, any detector capable of determining a property associated with the nanoscale wire can be used. The property can be electronic, electromagnetic, optical, mechanical, or the like. Examples of electrical or magnetic properties that can be determined include, but are not limited to, voltage, current, conductivity, resistance, impedance, inductance, charge, etc. Examples of optical properties associated with the nanoscale wire include its emission intensity, and/or emission wavelength, e.g. where the nanoscale wire is emissive. In some cases, the detector will include a power source and a metering device, for example a voltmeter or an ammeter.

In one embodiment, a conductance (or a change in conductance) less than 1 nS in a nanowire sensor of the invention can be detected. In another embodiment, a conductance in the range of thousandths of a nS can be detected. The concentration of a species, or analyte, may be detected from less than micromolar to molar concentrations and above. By using nanoscale wires with known detectors, sensitivity can be extended to a single molecules in some cases.

A variety of sample sizes, for exposure of a sample to a nanoscale sensor of the invention, can be used. As examples, the sample size used in nanoscale sensors may be less than or equal to about 10 microliters, less than or equal to about 1 microliter, or less than or equal to about 0.1 microliter. The sample size may be as small as about 10 nanoliters or less, in certain instances. The nanoscale sensor also allows for unique accessibility to biological species and may be used both in vivo and/or in vitro applications. When used in vivo, in some case, the nanoscale sensor and corresponding method result in a minimally invasive procedure.

The invention, in yet another set of embodiments, involves a sensing element comprising a sample exposure region and a nanoscale wire able to detect the presence or absence of an analyte, and/or the concentration of the analyte. The “sample exposure region” may be any region in close proximity to the nanoscale wire wherein a sample in the sample exposure region addresses at least a portion of the nanoscale wire. Examples of sample exposure regions include, but are not limited to, a well, a channel, a microchannel, and a gel. In certain embodiments, the sample exposure region is able to hold a sample proximate the nanoscale wire, and/or may direct a sample toward the nanoscale wire for determination of an analyte in the sample. The nanoscale wire may be positioned adjacent to or within the sample exposure region. Alternatively, the nanoscale wire may be a probe that is inserted into a fluid or fluid flow path. The nanoscale wire probe may also comprise a microneedle that supports and/or is integral with the nanoscale wire, and the sample exposure region may be addressable by the microneedle. In this arrangement, a device that is constructed and arranged for insertion of a microneedle probe into a sample can include a region surrounding or otherwise in contact with the microneedle that defines the sample exposure region, and a sample in the sample exposure region is addressable by the nanoscale wire, and vice versa. Fluid flow channels can be created at a size and scale advantageous for use in the invention (microchannels) using a variety of techniques such as those described in International Patent Application Serial No. PCT/US97/04005, entitled “Method of Forming Articles and Patterning Surfaces via Capillary Micromolding,” filed Mar. 14, 1997, published as Publication No. WO 97/33737 on Sep. 18, 1997, and incorporated herein by reference.

As an example, a sample, such as a fluid suspected of containing an analyte that is to be determined, may be presented to a sample exposure region of a sensing element comprising a nanoscale wire. An analyte present in the fluid that is able to bind to the nanoscale wire and/or a reaction entity immobilized relative to the nanoscale wire may cause a change in a property of the nanoscale wire that is determinable upon binding, e.g. using conventional electronics. If the analyte is not present in the fluid, the relevant property of the nanoscale wire will remain unchanged, and the detector will measure zero change. Thus, according to this particular example, the presence or absence of an analyte can be determined by monitoring changes, or lack thereof, in the property of the nanoscale wire.

In one set of embodiments, any of the techniques described herein may be used in the determination of proteins, small molecules, and the like, i.e., as in an assay. A property of an analyte may be determined by allowing the analyte to interact with a nanoscale wire and/or a reaction entity, and the interaction may be analyzed in some fashion, e.g., quantified. In some cases, the degree or amount of interaction (e.g., a binding constant) may be determined, for example, by measuring a property of the nanoscale wire (e.g., an electronic property, such as the conductance) after exposing the nanoscale wire and/or the reaction entity to the analyte.

In certain instances, such assays may be used in drug screening techniques. In one example, a protein or other target molecule may be immobilized relative to a nanoscale wire as a reaction entity, and exposed to one or more drug candidates, for example, serially or simultaneously. Interaction of the drug candidate(s) with the reaction entity may be determined by determining a property of the nanoscale wire, e.g., as previously described. As a non-limiting example, a nanoscale wire, having an associated target reaction entity, may be exposed to one or more species able to interact with the target reaction entity, for instance, the nanoscale wire may be exposed to a sample containing a first species able to interact with the target reaction entity, where the sample contains or is suspected of containing a second species able to interact with the target reaction entity, and optionally other, different species, where one of the species is a drug candidate. As one example, if the target reaction entity is an enzyme, the sample may contain a substrate and a drug candidate suspected of interacting with the enzyme in a way that inhibits enzyme/substrate interaction; if the target reaction entity is a substrate, the sample may contain an enzyme and a drug candidate suspected of interacting with the substrate in an inhibitory manner; if the target reaction entity is a nucleic acid, the sample may contain a complementary nucleic acid and a drug candidate suspected of interacting with the nucleic acid target reaction entity in an inhibitory manner; if the target reaction is a receptor, the sample may contain a ligand for the receptor and a drug candidate suspected of interacting with the receptor in an inhibitory manner; etc. In each of these cases, the drug candidate may act in a way that enhances, rather than inhibits, interaction.

In some cases, the assays of the invention may be used in high-throughput screening applications, e.g., where at least 100, at least 1,000, at least 10,000, or at least 100,000 or more analytes may be rapidly screened, for example, by exposing one or more analytes to a nanoscale wire (e.g., in solution), and/or exposing a plurality of analytes to a plurality of nanoscale wires and/or reaction entities.

In some embodiments, one or more nanoscale wires may be positioned in a microfluidic channel, which may define the sample exposure region in some cases. One or more different nanoscale wires may cross the same microfluidic channel (e.g., at different positions) to detect a different analyte, to measure a flowrate of an analyte(s), etc. In another embodiment, one or more nanoscale wires may be positioned in a microfluidic channel to form one of a plurality of analytic elements, for instance, in a microneedle probe, a dip and read probe, etc. The analytic elements probe may be implantable and capable of detecting several analytes simultaneously in real time, according to certain embodiments. In another embodiment, one or more nanowires may be positioned in a microfluidic channel to form an analytic elements in a microarray for a cassette or a lab on a chip device. Those skilled in the art would know such cassette or lab on a chip device will be in particular suitable for high throughout chemical analysis and screening, combinational drug discovery, etc. The ability to include multiple nanoscale wires in one nanoscale sensor also allows, in some cases, for the simultaneous detection of different analytes suspected of being present in a single sample. For example, a nanoscale pH sensor may include a plurality of nanoscale wires that each detect different pH levels, a nanoscale protein or nucleic acid sensor with multiple nanoscale wires may be used to detect multiple sequences, or combination of sequences, etc.

Thus, in one set of embodiments, an article of the invention may comprise a cassette comprising a sensing element having a sample exposure region and a nanoscale wire. The detection of an analyte in a sample within the sample exposure region may occur, in some cases, while the cassette is disconnected to a detector apparatus, allowing samples to be gathered at one site, and determined at another. The cassette may then be operatively connectable to a detector apparatus able to determine a property associated with the nanoscale wire. As used herein, a device is “operatively connectable” when it has the ability to attach and interact with another apparatus. In other cases, the cassette may be constructed and arranged such that samples may be gathered and determination at one site.

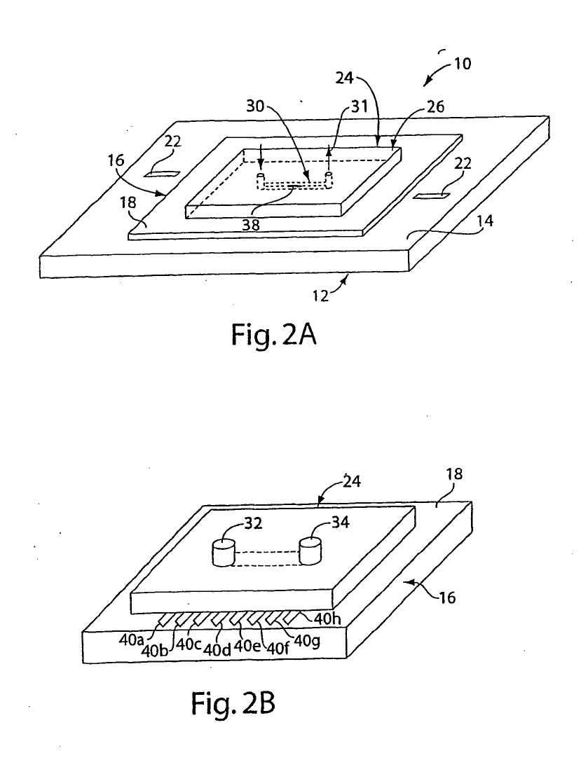

FIG. 2A shows one example of an article of the present invention where one or more nanoscale wires are positioned within a microfluidic channel. In FIG. 2A, nanoscale detector device 10 is comprised of a single nanowire 38 positioned above upper surface 18 of substrate 16. Chip carrier 12 has an upper surface 14 for supporting substrate 16 and electrical connections 22. Chip carrier 12, may be made of any insulating material that allows connection of electrical connections 22 to electrodes 36. In a preferred embodiment, the chip carrier is an epoxy. Upper surface 14 of the chip carrier, may be of any shape including, for example, planar, convex, and concave. In one embodiment, upper surface 14 of the chip carrier is planar.

As shown in FIG. 2A, lower surface of 20 of substrate 16 is positioned adjacent to upper surface 14 of the chip carrier and supports electrical connection 22. Substrate 16 may typically be made of a polymer, silicon, quartz, or glass, for example. In one embodiment, the substrate 16 is made of silicon coated with 600 nm of silicon oxide. Upper surface 18 and lower surface 20 of substrate 16 may be of any shape, such as planar, convex, and concave. In some cases, lower surface 20 of substrate 16 contours to upper surface 14 of chip carrier 12. Similarly, mold 24 has an upper surface 26 and a lower surface 28, either of which may be of any shape. In certain embodiments, lower surface 26 of mold 24 contours to upper surface 18 of substrate 16.

Mold 24 has a sample exposure region 30, shown here as a microchannel, having a fluid inlet 32 and fluid outlet 34, shown in FIG. 2A on the upper surface 26 of mold 24. Nanoscale wire 38 is positioned such that at least a portion of the nanoscale wire is positioned within sample exposure region 30. Electrodes 36 connect nanoscale wire 38 to electrical connection 22. Electrical connections 22 are, optionally, connected to a detector (not shown) that measures a change in an electrical, or other property of the nanoscale wire. The distance between electrodes 36 may range from 50 nm to about 20 microns, in some cases from about 100 nm to about 10 microns, or from about 500 nm to about 5 microns.

FIG. 2B shows another embodiment of the present invention wherein the nanoscale detector device 10 of FIG. 2A further includes multiple nanowires (not shown). In FIG. 2B, wire interconnects 40a-h connect to corresponding nanoscale wires to electrical connections, respectively (not shown). In some cases, each nanoscale wire has a unique reaction entity selected to detect a different analytes in the fluid. In this way, the determination (presence, absence, and/or amount) of several analytes may be determined using one sample while performing one test.

In one set of embodiments, an article of the invention is capable of delivering a stimulus to a nanoscale wire, and a detector is constructed and arranged to determine a signal resulting from the stimulus. For example, a nanoscale wire including a p-n junction can be delivered a stimulus (e.g., an electronic current), where the detector is constructed and arranged to determine a signal (e.g., electromagnetic radiation) resulting from the stimulus. In such an arrangement, an interaction of an analyte with the nanoscale wire, and/or with a reaction entity positioned proximate the nanoscale wire, can affect the signal in a detectable manner. In another example, where the reaction entity is a quantum dot, the quantum dot may be constructed to receive electromagnetic radiation of one wavelength and emit electromagnetic radiation of a different wavelength. Where the stimulus is electromagnetic radiation, it can be affected by interaction with an analyte, and the detector can detect a change in a signal resulting therefrom. Non-limiting examples of stimuli include a constant current/voltage, an alternating voltage, and electromagnetic radiation such as light.

In some embodiments, the sensing element may comprise a plurality of nanoscale wires able to determine (detect the presence, absence, and/or amount) of a plurality of one or more analytes. The individual nanoscale wires may be differentially doped as described herein, thereby varying the sensitivity of each nanoscale wires to the analyte. In some cases, individual nanoscale wires may be selected based on their ability to interact with specific analytes, thereby allowing the detection of a variety of analytes. The plurality of nanoscale wires may be randomly oriented or parallel to one another, according to another set of embodiments. The plurality of nanoscale wires may also be oriented in an array on a substrate in specific instances.

A sensing element of the present invention can collect real time data in some embodiments. The real time data may be used, for example, to monitor the reaction rate of a specific chemical or biological reaction. Physiological conditions or drug concentrations present in vivo may also produce a real time signal that may be used to control a drug delivery system. For example, the present invention includes, in one aspect, an integrated system, comprising a nanoscale wire detector, a reader and a computer controlled response system. In this example, the nanowire detects a change in the equilibrium of an analyte in the sample, feeding a signal to the computer controlled response system causing it to withhold or release a chemical or drug. This is particularly useful as an implantable drug or chemical delivery system because of its small size and low energy requirements. Those of ordinary skill in the art are well aware of the parameters and requirements for constructing implantable devices, readers, and computer-controlled response systems suitable for use in connection with the present invention. That is, the knowledge of those of ordinary skill in the art, coupled with the disclosure herein of nanowires as sensors, enables implantable devices, real-time measurement devices, integrated systems, and the like. Such systems can be made capable of monitoring one, or a plurality of physiological characteristics individually or simultaneously. Such physiological characteristics can include, for example, oxygen concentration, carbon dioxide concentration, glucose level, concentration of a particular drug, concentration of a particular drug by-product, or the like. Integrated physiological devices can be constructed to carry out a function depending upon a condition sensed by a sensor of the invention. For example, a nanowire sensor of the invention can sense glucose level and, based upon the determined glucose level can cause the release of insulin into a subject through an appropriate controller mechanism.

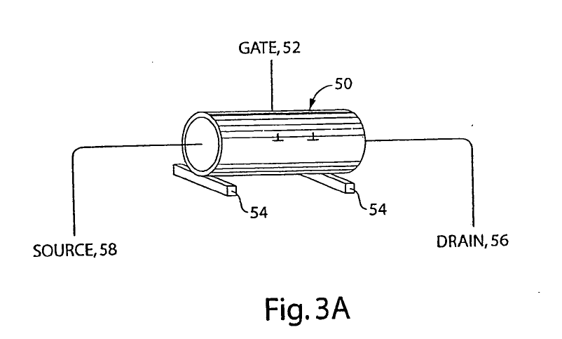

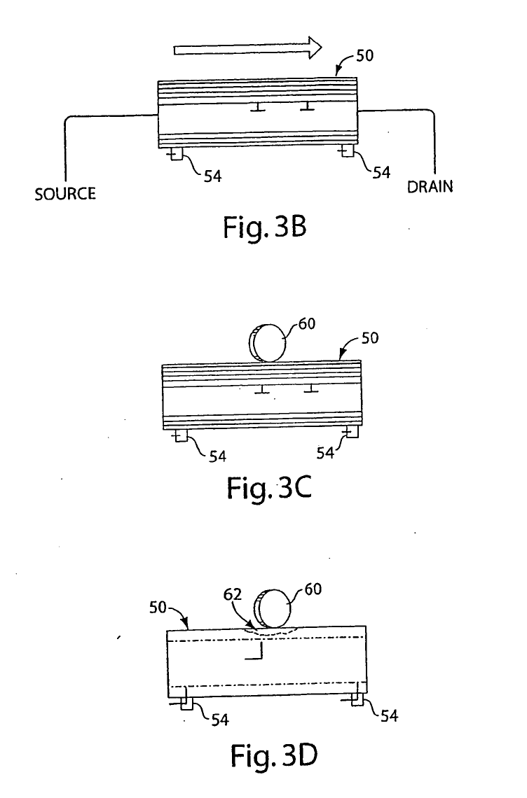

FIG. 3A depicts one example of an embodiment of a nanoscale wire sensor of the invention. In the embodiment shown in FIG. 3A, the nanoscale wire sensor invention comprises a single molecule of doped silicon 50. The doped silicon, as shown, is shaped as a tube in this particular example, and the doping can be n-doped or p-doped. The doped silicon nanoscale wire may form a high resistance semiconductor material across which a voltage may be applied. The exterior surface and/or the interior surface of the tube may have an oxide formed thereon. The surface of the tube can act as the gate 52 of an FET device and the electrical contacts at either end of the tube may allow the tube ends to acts as the drain 56 and the source 58. In the depicted embodiment the device is symmetric and either end of the device may be considered the drain or the source. For purpose of illustration, the nanoscale wire of FIG. 3A defines the left-hand side as the source and the right hand side as the drain. FIG. 3A also shows that the nanoscale wire device of this embodiment is disposed upon and electrically connected to two conductor elements 54.

FIGS. 3A and 3B illustrate an example of a chemical /or ligand-gated Field Effect Transistor (FET) that can define a sensor of the invention. FETs are well know in the art of electronics, and are described in more detail in, e.g., The Art of Electronics, Second Edition by Paul Horowitz and Winfield Hill, Cambridge University Press, 1989, pp. 113-174. In the FET, the availability of charge carriers is controlled by a voltage applied to a third “control electrode,” also known as the gate electrode. The conduction in the channel is controlled by a voltage applied to the gate electrode which produces an electric field across the channel. The device of FIGS. 3A and 3B may be considered a chemical or ligand-FET because the chemical or ligand provides the voltage at the gate which produced the electric field which changes the conductivity of the channel. This change in conductivity in the channel effects the flow of current through the channel. For this reason, a FET is often referred to as a transconductant device in which a voltage on the gate controls the current through the channel through the source and the drain. The gate of a FET is insulated from the conduction channel, for example, using a semiconductor junction such in a junction FET (JFET) or using an oxide insulator such as in a metal oxide semiconductor FET (MOSFET). Thus, in FIGS. 3A and 3B, the SiO₂ exterior surface of the nanoscale wire sensor may serve as the gate insulation for the gate.

In application, the nanoscale wire device illustrated in the example of FIG. 3 provides an FET device that may be contacted with a sample or disposed within the path of a sample flow. Analytes of interest within the sample can contact the surface of the nanoscale wire device and, under certain conditions, bind or otherwise adhere to the surface and/or affect the binding and/or adherence of other species. The exterior surface of the device may, in some cases, have reaction entities, e.g., binding partners that are specific for an analyte. The binding partners may attract the analyte and/or bind the analyte. An example is shown in FIG. 3C, where there is depicted an analyte 60 (not drawn to scale) bound to the surface of the nanoscale wire. Also shown, with reference to FIG. 3D, an analyte bound to the nanoscale wire may create a depletion region 62 within the nanoscale wire. In some cases, the depletion region may limit current passing through the wire. The depletion region can be depleted of holes or electrons, depending upon the type of channel. This is further shown schematically in FIG. 3D.

One aspect of the present invention includes a nanoscopic wire or other nanostructured material comprising one or more semiconductor and/or metal compounds, for example, for use in any of the above-described embodiments. In some cases, the semiconductors and/or metals may be chemically and/or physically combined, for example, as in a doped nanoscopic wire. The nanoscopic wire may be, for example, a nanorod, a nanowire, a nanowhisker, or a nanotube. The nanoscopic wire may be used in a device, for example, as a semiconductor component, a pathway, etc. The criteria for selection of nanoscale wires and other conductors or semiconductors for use in the invention are based, in some instances, upon whether the nanoscale wire is able to interact with an analyte, or whether the appropriate reaction entity, e.g. a binding partner, can be easily attached to the surface of the nanoscale wire, or the appropriate reaction entity, e.g. a binding partner, is near the surface of the nanoscale wire. Selection of suitable conductors or semiconductors, including nanoscale wires, will be apparent and readily reproducible by those of ordinary skill in the art with the benefit of the present disclosure.

Examples of nanotubes that may be used in the present invention include, but are not limited to, single-walled nanotubes (SWNTs). Structurally, SWNTs are formed of a single graphene sheet rolled into a seamless tube. Depending on the diameter and helicity, SWNTs can behave as one-dimensional metals and/or semiconductors. SWNTs. Methods of manufacture of nanotubes, including SWNTs, and characterization are known. Methods of selective functionalization on the ends and/or sides of nanotubes also are known, and the present invention makes use of these capabilities for molecular electronics in certain embodiments. Multi-walled nanotubes are well known, and can be used as well.

Many nanoscopic wires as used in accordance with the present invention are individual nanoscopic wires. As used herein, “individual nanoscopic wire” means a nanoscopic wire free of contact with another nanoscopic wire (but not excluding contact of a type that may be desired between individual nanoscopic wires, e.g., as in a crossbar . array). For example, an “individual” or a “free-standing” article may, at some point in its life, not be attached to another article, for example, with another nanoscopic wire, or the free-standing article may be in solution. This is in contrast to nanotubes produced primarily by laser vaporization techniques that produce materials formed as ropes having diameters of about 2 nm to about 50 nm or more and containing many individual nanotubes. This is also in contrast to conductive portions of articles which differ from surrounding material only by having been altered chemically or physically, in situ, i.e., where a portion of a uniform article is made different from its surroundings by selective doping, etching, etc. An “individual” or a “free-standing” article is one that can be (but need not be) removed from the location where it is made, as an individual article, and transported to a different location and combined with different components to make a functional device such as those described herein and those that would be contemplated by those of ordinary skill in the art upon reading this disclosure.

In another set of embodiments, the nanoscopic wire (or other nanostructured material) may include additional materials, such as semiconductor materials, dopants, organic compounds, inorganic compounds, etc. The following are non-limiting examples of materials that may be used as dopants within the nanoscopic wire. The dopant may be an elemental semiconductor, for example, silicon, germanium, tin, selenium, tellurium, boron, diamond, or phosphorous. The dopant may also be a solid solution of various elemental semiconductors. Examples include a mixture of boron and carbon, a mixture of boron and P(BP₆), a mixture of boron and silicon, a mixture of silicon and carbon, a mixture of silicon and germanium, a mixture of silicon and tin, a mixture of germanium and tin, etc. In some embodiments, the dopant may include mixtures of Group IV elements, for example, a mixture of silicon and carbon, or a mixture of silicon and germanium. In other embodiments, the dopant may include mixtures of Group III and Group V elements, for example, BN, BP, BAs, AlN, AlP, AlAs, AlSb, GaN, GaP, GaAs, GaSb, InN, InP, InAs, or InSb. Mixtures of these combinations may also be used, for example, a mixture of BN/BP/BAs, or BN/AlP. In other embodiments, the dopants may include mixtures of Group III and Group V elements. For example, the mixtures may include AlGaN, GaPAs, InPAs, GaInN, AlGaInN, GaInAsP, or the like. In other embodiments, the dopants may also include mixtures of Group II and Group VI elements. For example, the dopant may include mixtures of ZnO, ZnS, ZnSe, ZnTe, CdS, CdSe, CdTe, HgS, HgSe, HgTe, BeS, BeSe, BeTe, MgS, MgSe, or the like. Alloys or mixtures of these dopants are also be possible, for example, ZnCd Se, or ZnSSe or the like. Additionally, mixtures of different groups of semiconductors may also be possible, for example, combinations of Group II-Group VI and Group III-Group V elements, such as (GaAs)ₓ(ZnS)₁₋ₓ. Other non-limiting examples of dopants may include mixtures of Group IV and Group VI elements, for example GeS, GeSe, GeTe, SnS, SnSe, SnTe, PbO, PbS, PbSe, PbTe, etc. Other dopant mixtures may include mixtures of Group I elements and Group VII elements, such as CuF, CuCl, CuBr, CuI, AgF, AgCl, AgBr, AgI, or the like. Other dopant mixtures may include different mixtures of these elements, such as BeSiN₂, CaCN₂, ZnGeP₂, CdSnAs₂, ZnSnSb₂, CuGeP₃, CuSi₂P₃, Si₃N₄, Ge₃N₄, Al₂O₃, (Al, Ga, In)₂(S, Se, Te)₃, Al₂CO, (Cu, Ag)(Al, Ga, In, Tl, Fe)(S, Se, Te)₂ or the like.

As a non-limiting example, a p-type dopant may be selected from Group III, and an n-type dopant may be selected from Group V. For instance, a p-type dopant may include at least one of B, Al and In, and an n-type dopant may include at least one of P, As and Sb. For Group III-Group V mixtures, a p-type dopant may be selected from Group II, including one or more of Mg, Zn, Cd and Hg, or Group IV, including one or more of C and Si. An n-type dopant may be selected from at least one of Si, Ge, Sn, S, Se and Te. It will be understood that the invention is not limited to these dopants, but may include other elements, alloys, or mixtures as well.

As used herein, the tern “Group,” with reference to the Periodic Table, is given its usual definition as understood by one of ordinary skill in the art. For instance, the Group II elements include Mg and Ca, as well as the Group II transition elements, such as Zn, Cd, and Hg. Similarly, the Group III elements include B, Al, Ga, In and TI; the Group IV elements include C, Si, Ge, Sn, and Pb; the Group V elements include N, P, As, Sb and Bi; and the Group VI elements include O, S, Se, Te and Po. Combinations involving more than one element from each Group are also possible. For example, a Group II-VI material may include at least one element from Group II and at least one element from Group VI, e.g., ZnS, ZnSe, ZnSSe, ZnCdS, CdS, or CdSe. Similarly, a Group III-V material may include at least one element from Group III and at least one element from Group V, for example GaAs, GaP, GaAsP, InAs, InP, AlGaAs, or InAsP. Other dopants may also be included with these materials and combinations thereof, for example, transition metals such as Fe, Co, Te, Au, and the like. The nanoscale wire of the present invention may further include, in some cases, any organic or inorganic molecules. In some cases, the organic or inorganic molecules are polarizable and/or have multiple charge states.

In some embodiments, at least a portion of a nanoscopic wire may be a bulk-doped semiconductor. As used herein, a “bulk-doped” article (e. g. an article, or a section or region of an article) is an article for which a dopant is incorporated substantially throughout the crystalline lattice of the article, as opposed to an article in which a dopant is only incorporated in particular regions of the crystal lattice at the atomic scale, for example, only on the surface or exterior. For example, some articles such as carbon nanotubes are typically doped after the base material is grown, and thus the dopant only extends a finite distance from the surface or exterior into the interior of the crystalline lattice. It should be understood that “bulk-doped” does not define or reflect a concentration or amount of doping in a semiconductor, nor does it necessarily indicate that the doping is uniform. In particular, in some embodiments, a bulk-doped semiconductor may comprise two or more bulk-doped regions. Thus, as used herein to describe nanoscopic wires, “doped” refers to bulk-doped nanoscopic wires, and, accordingly, a “doped nanoscopic (or nanoscale) wire” is a bulk-doped nanoscopic wire. “Heavily doped” and “lightly doped” are terms the meanings of which are clearly understood by those of ordinary skill in the art.

In one set of embodiments, the invention includes a nanoscale wire (or other nanostructured material) that is a single crystal. As used herein, a “single crystal” item (e.g., a semiconductor) is an item that has covalent bonding, ionic bonding, or a combination thereof throughout the item. Such a single-crystal item may include defects in the crystal, but is to be distinguished from an item that includes one or more crystals, not ionically or covalently bonded, but merely in close proximity to one another.

In yet another set of embodiments, the nanoscale wire (or other nanostructured material) may comprise two or more regions having different compositions. Each region of the nanoscale wire may have any shape or dimension, and these can be the same or different between regions. For example, a region may have a smallest dimension of less than 1 micron, less than 100 nm, less than 10 nm, or less than 1 nm. In some cases, one or more regions may be a single monolayer of atoms (i.e., “delta-doping”). In certain cases, the region may be less than a single monolayer thick (for example, if some of the atoms within the monolayer are absent).

The two or more regions may be longitudinally arranged relative to each other, and/or radially arranged (e.g., as in a core/shell arrangement) within the nanoscale wire. As one example, the nanoscale wire may have multiple regions of semiconductor materials arranged longitudinally. In another example, a nanoscale wire may have two regions having different compositions arranged longitudinally, surrounded by a third region or several regions, each having a composition different from that of the other regions. As a specific example, the regions may be arranged in a layered structure within the nanoscale wire, and one or more of the regions may be delta-doped or at least partially delta-doped. As another example, the nanoscale wire may have a series of regions positioned both longitudinally and radially relative to each other. The arrangement can include a core that differs in composition along its length (changes in composition or concentration longitudinally), while the lateral (radial) dimensions of the core do, or do not, change over the portion of the length differing in composition. The shell portions can be adjacent each other (contacting each other, or defining a change in composition or concentration of a unitary shell structure longitudinally), or can be separated from each other by, for example, air, an insulator, a fluid, or an auxiliary, non-nanoscale wire component. The shell portions can be positioned directly on the core, or can be separated from the core by one or more intermediate shells portions that can themselves be constant in composition longitudinally, or varying in composition longitudinally, i.e., the invention allows the provision of any combination of a nanowire core and any number of radially-positioned shells (e.g., concentric shells), where the core and/or any shells can vary in composition and/or concentration longitudinally, any shell sections can be spaced from any other shell sections longitudinally, and different numbers of shells can be provided at different locations longitudinally along the structure.

In still another set of embodiments, a nanoscale wire may be positioned proximate the surface of a substrate, i.e., the nanoscale wire may be positioned within about 50 nm, about 25 nm, about 10 nm, or about 5 nm of the substrate. In some cases, the proximate nanoscale wire may contact at least a portion of the substrate. In one embodiment, the substrate comprises a semiconductor and/or a metal. Non-limiting examples include Si, Ge, GaAs, etc. Other suitable semiconductors and/or metals are described above with reference to nanoscale wires. In certain embodiments, the substrate may comprise a nonmetal/nonsemiconductor material, for example, a glass, a plastic or a polymer, a gel, a thin film, etc. Non-limiting examples of suitable polymers that may form or be included in the substrate include polyethylene, polypropylene, poly(ethylene terephthalate), polydimethylsiloxane, or the like.

In certain aspects, the present invention provides a method of preparing a nanostructure. In one set of embodiments, the method involves allowing a first material to diffuse into at least part of a second material, optionally creating a new compound. For example, the first and second materials may each be metals or semiconductors, one material may be a metal and the other material may be a semiconductor, etc. In certain embodiments, the present invention involves controlling and altering the doping of semiconductors in a nanoscale wire. In some cases, the nanoscale wires (or other nanostructure) may be produced using techniques that allow for direct and controlled growth of the nanoscale wires. In some cases, the nanoscale wire may be doped during growth of the nanoscale wire. Doping the nanoscale wire during growth may result in the property that the doped nanoscale wire is bulk-doped. Furthermore, such doped nanoscale wires may be controllably doped, such that a concentration of a dopant within the doped nanoscale wire can be controlled and therefore reproduced consistently.

Certain arrangements may utilize metal-catalyzed CVD techniques (“chemical vapor deposition”) to synthesize individual nanoscale wires. CVD synthetic procedures useful for preparing individual wires directly on surfaces and in bulk form are generally known, and can readily be carried out by those of ordinary skill in the art. Nanoscopic wires may also be grown through laser catalytic growth. With the same basic principles as LCG, if uniform diameter nanoclusters (less than 10% to 20% variation depending on how uniform the nanoclusters are) are used as the catalytic cluster, nanoscale wires with uniform size (diameter) distribution can be produced, where the diameter of the wires is determined by the size of the catalytic clusters. By controlling growth time, nanoscale wires with different lengths can be grown.

One technique that may be used to grow nanoscale wires is catalytic chemical vapor deposition (“C-CVD”). In C-CVD, reactant molecules are formed from the vapor phase. Nanoscale wires may be doped by introducing the doping element into the vapor phase reactant (e.g. diborane and phosphane). The doping concentration may be controlled by controlling the relative amount of the doping compound introduced in the composite target. The final doping concentration or ratios are not necessarily the same as the vapor-phase concentration or ratios. By controlling growth conditions, such as temperature, pressure or the like, nanoscale wires having the same doping concentration may be produced.

Another technique for direct fabrication of nanoscale wire junctions during synthesis is referred to as laser catalytic growth (“LCG”). In LCG, dopants are controllably introduced during vapor phase growth of nanoscale wires. Laser vaporization of a composite target composed of a desired material (e.g. silicon or indium phosphide) and a catalytic material (e.g. a nanoparticle catalyst) may create a hot, dense vapor. The vapor may condense into liquid nanoclusters through collision with a buffer gas. Growth may begin when the liquid nanoclusters become supersaturated with the desired phase and can continue as long as reactant is available. Growth may terminate when the nanoscale wire passes out of the hot reaction zone and/or when the temperature is decreased. The nanoscale wire may be further subjected to different semiconductor reagents during growth.

Other techniques to produce nanoscale semiconductors such as nanoscale wires are also contemplated. For example, nanoscale wires of any of a variety of materials may be grown directly from vapor phase through a vapor-solid process. Also, nanoscale wires may also be produced by deposition on the edge of surface steps, or other types of patterned surfaces. Further, nanoscale wires may be grown by vapor deposition in or on any generally elongated template. The porous membrane may be porous silicon, anodic alumina, a diblock copolymer, or any other similar structure. The natural fiber may be DNA molecules, protein molecules carbon nanotubes, any other elongated structures. For all the above described techniques, the source materials may be a solution or a vapor. In some cases, while in solution phase, the template may also include be column micelles formed by surfactant.

In some cases, the nanoscale wire may be doped after formation. In one technique, a nanoscale wire having a substantially homogeneous composition is first synthesized, then is doped post-synthetically with various dopants. Such doping may occur throughout the entire nanoscale wire, or in one or more portions of the nanoscale wire, for example, in a wire having multiple regions differing in composition.

One aspect of the invention provides for the assembly, or controlled placement, of nanoscale wires on a surface. Any substrate may be used for nanoscale wire placement, for example, a substrate comprising a semiconductor, a substrate comprising a metal, a substrate comprising a glass, a substrate comprising a polymer, a substrate comprising a gel, a substrate that is a thin film, a substantially transparent substrate, a non-planar substrate, a flexible substrate, a curved substrate, etc. In some cases, assembly can be carried out by aligning nanoscale wires using an electrical field. In other cases, assembly can be performed using an arrangement involving positioning a fluid flow directing apparatus to direct fluid containing suspended nanoscale wires toward and in the direction of alignment with locations at which nanoscale wires are desirably positioned.

In certain cases, a nanoscale wire (or other nanostructure) is formed on the surface of a substrate, and/or is defined by a feature on a substrate. In one example, a nanostructure, such as a nanoscale wire, is formed as follows. A substrate is imprinted using a stamp or other applicator to define a pattern, such as a nanoscale wire or other nanoscale structure. After removal of the stamp or other applicator, at least a portion of the imprintable layer is removed, for example, through etching processes such as reactive ion etching (RIE), or other known techniques. In some cases, enough imprintable material may be removed from the substrate so as to expose portions of the substrate free of the imprintable material. A metal or other materials may then be deposited onto at least a portion of the substrate, for example, gold, copper, silver, chromium, etc. In some cases, a “lift-off” step may then be performed, where at least a portion of the imprintable material is removed from the substrate. Metal or other material deposited onto the imprintable material may be removed along with the removal of the imprintable material, for example, to form one or more nanoscale wires. Structures deposited on the surface may be connected to one or more electrodes in some cases. The substrate may be any suitable substrate that can support an imprintable layer, for example, comprising a semiconductor, a metal, a glass, a polymer, a gel, etc. In some cases, the substrate may be a thin film, substantially transparent, non-planar, flexible, and/or curved, etc.

In certain cases, an array of nanowires may be produced by providing a surface having a plurality of substantially aligned nanoscale wires, and removing, from the surface, a portion of one or more of the plurality of nanoscale wires. The remaining nanoscale wires on the surface may then be connected to one or more electrodes. In certain cases, the nanoscopic wires are arranged such that they are in contact with each other; in other instances, however, the aligned nanoscopic wires may be at a pitch such that they are substantially not in physical contact.

In certain cases, nanoscale wires are positioned proximate a surface using flow techniques, i.e., techniques where one or more nanoscale wires may be carried by a fluid to a substrate. Nanoseale wires (or any other elongated structures) can be aligned by inducing a flow of a nanoscale wire solution on surface, where the flow can include channel flow or flow by any other suitable technique. Nanoscale wire arrays with controlled position and periodicity can be produced by patterning a surface of a substrate and/or conditioning the surface of the nanoscale wires with different functionalities, where the position and periodicity control may be achieved by designing specific complementary forces between the patterned surface and the nanoscale wires. Nanoscale wires can also be assembled using a Langmuir-Blodgett (LB) trough. Nanoscale wires may first be surface-conditioned and dispersed to the surface of a liquid phase to form a Langmuir-Blodgett film. In some cases, the liquid may include a surfactant, which can, in some cases, reduce aggregation of the nanoscale wires and/or reduce the ability of the nanoscale wires to interact with each other. The nanoscale wires can be aligned into different patterns (such as parallel arrays or fibers) by compressing the surface or reducing the surface area of the surface.

Another arrangement involves forming surfaces on a substrate including regions that selectively attract nanoscale wires surrounded by regions that do not selectively attract them. Surfaces can be patterned using known techniques such as electron-beam patterning, “soft-lithography” such as that described in International Patent Application Serial No. PCT/US96/03073, entitled “Microcontact Printing on Surfaces and Derivative Articles,” filed Mar. 1, 1996, published as Publication No. WO 96/29629 on Jul. 26, 1996; or U.S. Pat. No. 5,512,131, entitled “Formation of Microstamped Patterns on Surfaces and Derivative Articles,” issued Apr. 30, 1996, each of which is incorporated herein by reference. Additional techniques are described in U.S. Patent Application Serial No. 60/142,216, entitled “Molecular Wire-Based Devices and Methods of Their Manufacture,” filed Jul. 2, 1999, incorporated herein by reference. Fluid flow channels can be created at a size scale advantageous for placement of nanoscale wires on surfaces using a variety of techniques such as those described in International Patent Application Serial No. PCT/US97/04005, entitled “Method of Forming Articles and Patterning Surfaces via Capillary Micromolding,” filed Mar. 14, 1997, published as Publication No. WO 97/33737 on Sep. 18, 1997, and incorporated herein by reference. Other techniques include those described in U.S. Pat. No. 6,645,432, entitled “Microfluidic Systems Including Three-dimensionally Arrayed Channel Networks,” issued Nov. 11, 2003, incorporated herein by reference.

Chemically patterned surfaces other than SAM-derivatized surfaces can be used, and many techniques for chemically patterning surfaces are known. Another example of a chemically patterned surface may be a micro-phase separated block copolymer structure. These structures may provide a stack of dense lamellar phases, where a cut through these phases reveals a series of “lanes” wherein each lane represents a single layer. The assembly of nanoscale wires onto substrate and electrodes can also be assisted using bimolecular recognition in some cases. For example, one biological binding partner may be immobilized onto the nanoscale wire surface and the other one onto a substrate or an electrode using physical adsorption or covalently linking. An example technique which may be used to direct the assembly of a nanoscopic wires on a substrate is by using “SAMs,” or self-assembled monolayers. Any of a variety of substrates and SAM-forming material can be used along with microcontact printing techniques, such as those described in International Patent Application Serial No. PCT/US96/03073, entitled “Microcontact Printing on Surfaces and Derivative Articles,” filed Mar. 1, 1996, published as Publication No. WO 96/29629 on Jul. 26, 1996, incorporated herein by reference in its entirety.

In some cases, the nanoscale wire arrays may also be transferred to another substrate, e.g., by using stamping techniques. In certain instances, nanoscale wires may be assembled using complementary interaction, i.e., where one or more complementary chemical, biological, electrostatic, magnetic or optical interactions are used to position one or more nanoscale wires on a substrate. In certain cases, physical patterns may be used to position nanoscale wires proximate a surface. For example, nanoscale wires may be positioned on a substrate using physical patterns, for instance, aligning the nanoscale wires using corner of the surface steps or along trenches on the substrate.

The following examples are intended to illustrate certain aspects of certain embodiments of the present invention, but do not exemplify the full scope of the invention.

### EXAMPLE 1

The development of miniaturized devices for sensing the specific binding of small molecules to proteins is of substantial importance to the discovery and screening of new drug molecules. This example demonstrates highly sensitive, label-free, real-time detection of small molecule inhibitors of ATP binding to Abl, a protein tyrosine kinase whose constitutive activity is responsible for chronic myelogenous leukemia. In this example, Abl protein was covalently linked to the surfaces of a silicon nanowire field-effect device, and then concentration-dependent binding of ATP and concentration-dependent inhibition of ATP binding by the competitive small-molecule antagonist STI-571 (Gleevec or “Gle”) were assessed by monitoring the nanowire conductance. This example also demonstrates that the nanowire sensor can readily distinguish the affinities of distinct small molecule inhibitors and thus could serve as a new technology platform for drug discovery.

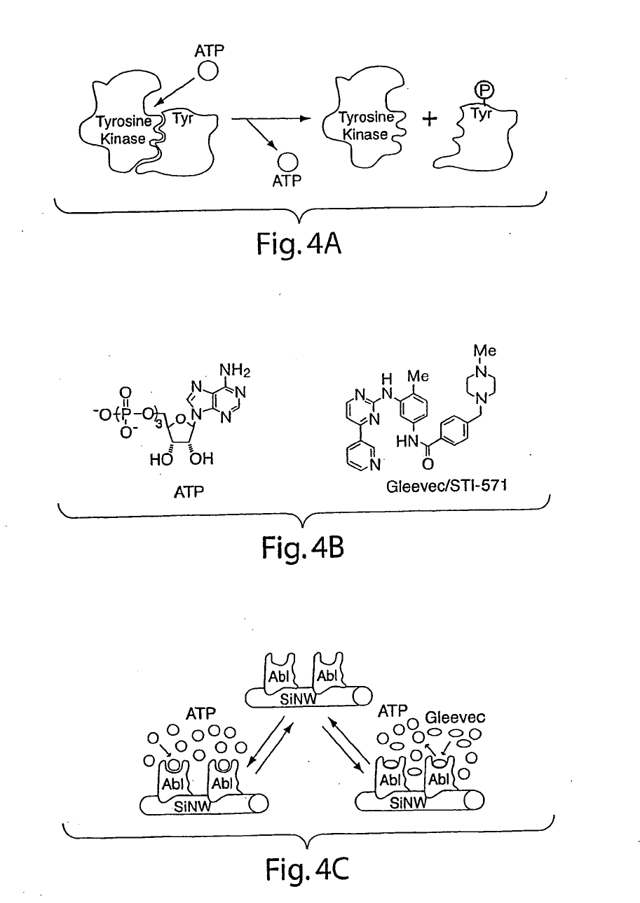

The identification of organic molecules that bind specifically to proteins is central to the discovery and development of new pharmaceuticals and to chemical genetic approaches for elucidating complex pathways in biological systems. Broadly representative of the importance of this concept for developing drugs to treat disease has been efforts focused on identifying inhibitors to protein tyrosine kinases. Tyrosine kinases represent attractive targets since they are central elements in the networks that mediate signal transduction in mammalian cells. The regulatory function of tyrosine kinases occurs through phosphorylation of a tyrosine residue of a substrate protein using adenosine triphosphate (ATP) as a phosphate source (FIG. 4A), and the subsequent transmission of this event through signal transduction cascade. Deregulation of phosphorylation through, for example, mutation or overexpression of protein tyrosine kinases, has been linked to a number of diseases including cancer. FIG. 4A illustrates the basic activity of a tyrosine kinase, where ATP binds to the tyrosine kinase active site, and then the gamma-phosphate group is transferred to tyrosine (Tyr) residue of the substrate protein.

The identification of inhibitors to ATP or substrate protein binding can thus serve as a means of treating diseases linked to a tyrosine kinase. A successful example of this strategy has been the introduction of the small molecule STI-571 or Gleevec (FIG. 4B), which competitively inhibits ATP binding to the tyrosine kinase Abl and is a highly effective treatment for chronic myelogenous leukemia, CML. This success and the recognition that Gleevec may be unable to cure late stage CML due to mutations in the kinase suggest that the development of approaches that enable rapid, flexible and quantitative comparison of small molecule inhibitors of ATP or substrate protein binding to tyrosine kinases, including those with mutations, could substantially improve drug discovery and development. In this example, a highly sensitive detection scheme for identifying small molecule inhibitors is demonstrated that does not require labeling of the protein, ATP or small molecule and can be carried out in real-time.

To develop a general system for screening small molecule inhibitors to tyrosine kinases the Abl kinase was linked to the surface of SiNW (silicon nanowire) FETs and investigated the binding of ATP and competitive inhibition of ATP binding with organic molecules (FIG. 4C). FIG. 4C illustrates the detection of ATP binding and small molecule inhibition of binding using a SiNW sensor device. The tyrosine kinase Abl was covalently linked to the surface of a SiNW and then the conductance of the nanowire device was monitored to detect ATP binding and the competitive inhibition of ATP binding by Gleevec. In this way, it was possible to monitor in real-time the binding or inhibition of binding of the negatively charged ATP to Abl as a conductance change due to chemical gating.

SiNW FETs were prepared using procedures similar to those described above. It was shown that the SiNW FETs exhibited reproducible electronic characteristics and a surface oxide, SiO₂, that was compatible with chemistry developed for the efficient linkage of proteins to glass chips. The Abl protein was covalently-linked through lysine residues to SiNW FETs within an integrated microfluidic channel, washed with buffer and used without further modification or dehydration. The binding experiments were carried out in buffered solutions with ionic strengths 10-1000 times greater than the ATP or small molecule inhibitor concentrations.

The SiNWs were prepared as follows. Bare SiNWs (in the form of nanowire FETs) were cleaned by oxygen plasma (0.3 Torr, 25 W power for 60 s) to remove contaminants, then immersed into an ethanol solution containing 2% aldehyde propyltrimethoxysilane (United 11 Chemical Technologies, Philadelphia, Pa.), 4% water, and 0.1% acetic acid for 1 hour, followed by thorough rinsing with 100% ethanol and baking at 120° C. for 10 min in an N₂ atmosphere to terminate the nanowire surface with aldehyde groups. Microfluidic channels (200 micron height and width) made using PDMS (polydimethoxysiloxane) molds and pre-coated with polyethylene glycol (MW 5000, Shearwater, Huntsville, Ala.) to reduce unspecific adsorption of proteins were aligned precisely onto aldehyde-terminated nanowires. Prior to coupling, the Abl tyrosine kinase solution, purchased from New England Biolabs (Berverly, Mass.), was dialyzed against 15 mM HEPES buffer at pH=7.5 containing 0.1 mM MgCl₂ and 0.1 mM EGTA (surface functionalization buffer) with a MINI dialysis unit purchased from Pierce (Rockford, Ill.).

A small amount of sodium cyanoborohydride (Aldrich, Boston, Mass.) was added to the dialyzed Abl tyrosine kinase solution. The Abl tyrosine kinase was then coupled onto the SiNW surface by flowing the kinase through the microfluidic channel at a concentration of 5 micrograms/ml at a flow rate of 0.15 ml/hr. After the coupling reaction was completed, 15 mM of tris buffer was flowed through the channel for 5 to 10 min to quench unreacted aldehyde groups. Immediately before the measurement, a measurement buffer (1.5 micromolar HEPES buffer at pH 7.5 containing 1 micromolar MgCl₂ and 1 micromolar EGTA) was flowed through the sensor surface to establish a baseline.

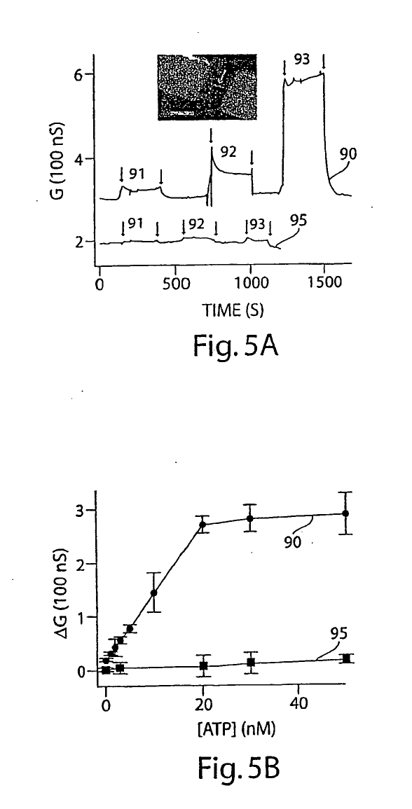

Typical time-dependent data recorded from an Abl modified SiNW device (shown in FIG. 5A) exhibited reversible, concentration-dependent increases in conductance upon introducing solutions containing ATP. FIG. 5A shows conductance (G) vs. ATP concentration for SiNWs modified with Abl (90) and a device prepared in an identical fashion except Abl was not coupled to the surface (95). Regions 91, 92 and 93 correspond to 0.1, 3, and 20 nM ATP, respectively. Arrows indicate the points where the solution was changed. The conductance of SiNW FETs was recorded using lock-in amplifier at 31 Hz and 30 mV modulation amplitude; the dc-bias voltage was zero. The inset in FIG. 5A is a scanning electron micrograph of a typical SiNW FET device. The nanowire is highlighted by a white arrow and is contacted on either end with Ti/Au metal electrodes. The scale bar is 500 nm. ATP was dissolved in 1.5 micromolar HEPES buffer containing 1 micromolar MgCl₂ and 1 micromolar EGTA. The flow rate was kept constant at 0.2 ml/hr.

The conductance changes exhibited some variations versus time after switching between buffer and buffer+ATP (inhibitor) solutions; for example, between sets of arrows in FIG. 5A. These variations were believed to arise from electrical noise produced when solution reservoirs are switched (short time scales), and sampling sites with different accessibility at longer time scales.

The observed increases in conductance were consistent with that expected for negatively charged ATP binding to Abl, since the negative charge will lead to accumulation of carriers in the p-type SiNW. The p-type SiNW FETs exhibited an increase (decrease) in conductance when gate voltage was negative (positive) due to the accumulation (depletion) of carriers. The binding of negatively charge ATP to the Abl kinase increased the negative surface charge density and increased conductance similar to a negative gate voltage. Control experiments carried out with devices prepared in the same manner, except that Abl protein was not coupled to the surfaces, showed little or no change in conductance upon addition of the same concentration ATP solutions. These experiments thus demonstrated that the conductance changes observed for the Abl-modified SiNW devices corresponded to specific binding of ATP to the tyrosine kinase.

The data also showed that the addition of pure buffer solution following ATP binding resulted in a decrease in the device conductance to the baseline value independent of the ATP concentration, i.e., binding and detection were reversible as expected. In addition, the data demonstrated that ATP binding to Abl could be readily distinguished above background at concentrations at least as low as 100 pM. Plots summarizing the concentration-dependent ATP binding to Abl monitored by the SiNW devices exhibited a characteristic linear response at low concentrations and saturation at higher concentrations (FIG. 5B); however, devices without Abl linked to the surface showed essentially no response. FIG. 5B shows the change in conductance (delta-G or ΔG) vs. ATP concentration for Abl-modified SiNW (90) and SiNW without Abl (95). The devices were fabricated by dispersing boron-doped SiNWs on degenerately doped silicon wafers with 600 nm oxide, followed by electron beam lithography and electron beam evaporation to make Ti (60 nm) and Au (40 nm) metal contacts. The ATP binding constant was estimated from the linear response region of the data to be about 100 nM. The ATP dissociation constant estimated from the linear response region was about 100 nM.

The ability to rapidly quantify ATP binding without specific labels using these SiNW devices contrasts conventional assays in which the incorporation of radioactive ³²P from labeled ATP is monitored following autophosphorylation or reaction with substrate. Thus this system may be used as a simple and quantitative screen for ATP binding to proteins.

### EXAMPLE 2

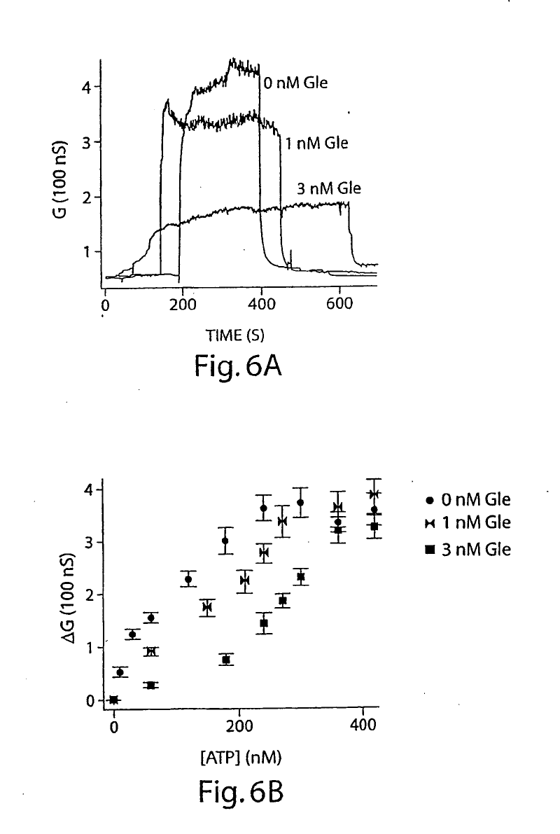

This example demonstrates the use of certain SiNW devices of the invention to monitor directly competitive inhibition of ATP binding by small molecules. Measurements made using the Abl modified SiNW devices, as described above with reference to Example 1 demonstrated that the conductance changed as a function of varying concentration of the inhibitor Gleevec was introduced to solutions of fixed ATP concentration. Specifically, increases in the Gleevec concentration at fixed ATP concentration yielded decreases in the conductance change associated with ATP binding (FIG. 6A), that is, Gleevec competes with ATP for the binding site in Abl. Notably, these results demonstrated that this approach provides facile, label-free detection of small molecule inhibition. FIG. 6A illustrates the conductance vs. time data for ATP binding in the presence of different concentrations of Gleevec. The ATP concentration was fixed at 240 nM in the three experiments. ATP and Gleevec solutions were made in the same buffer as described in Example 1.

FIG. 6B illustrates the change in conductance (delta-G or ΔG) vs. ATP concentration for Abl-modified SiNW in the presence of different base concentrations of Gleevec. The concentrations are as indicated. Measurements of the conductance changes as a function of ATP concentration for two fixed concentrations of Gleevec (FIG. 6B) demonstrated several key points. First, the ATP binding curves were found to have shifted systematically to the right (higher ATP concentration) as Gleevec was increased from 1 to 3 nM, although the saturation conductance changes at high ATP concentrations were very similar. These results are consistent with reversible competitive inhibition of an agonist (ATP) with an antagonist (Gleevec). The presence of Gleevec reduced the total number of available binding sites at relatively low ATP concentrations, and this effectively translates into lower sensor response at a fixed ATP concentration. However, sufficiently high ATP concentrations overwhelmed the influence of Gleevec, thus, a saturation response due to total receptor occupancy was ultimately observed. Second, these data can be used to provide a quantitative measure of Gleevec inhibition to ATP binding. The shift in the ATP binding curves in FIG. 6B could be analyzed using the equation C′/C=1+[I]/K_{I}, where C and C′ are the concentrations of ATP required to produce a conductance response in the absence and presence, respectively, of inhibitor at [I], and K_{I} is the inhibition constant. Analysis of this data yielded a K_{I} of about 2 nM, similar to, but smaller than, the value obtained from kinetic assays.

### EXAMPLE 3

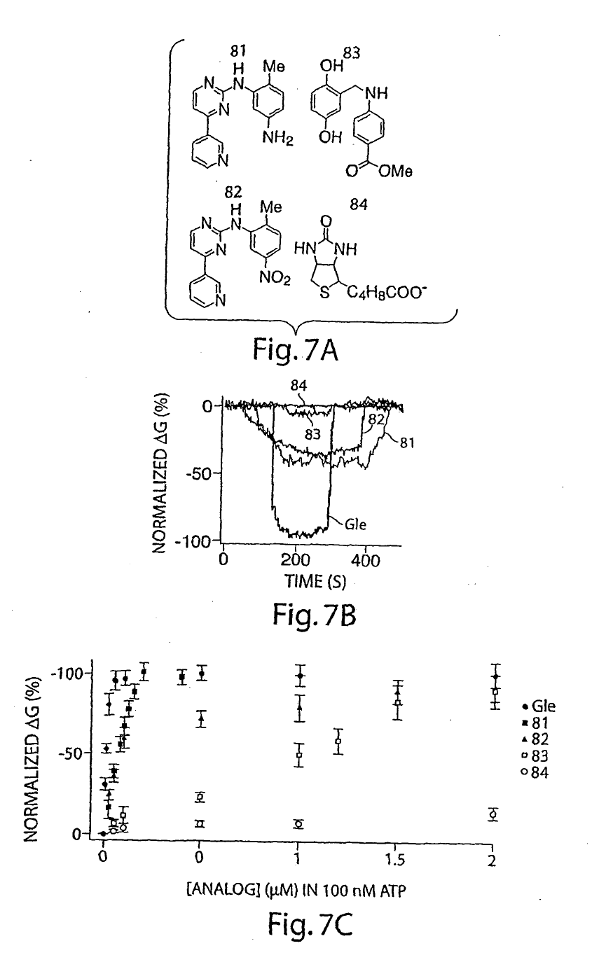

The results of Example 2 show rapid and direct screening of small molecule inhibitors of ATP binding in tyrosine kinases using the SiNW detectors. In this example, the ATP binding by four additional small molecules, two of which are known inhibitors for Abl, was investigated. Molecules 81, 82, and 83 have structural homology with Gleevec, while the fourth molecule tested, biotin 84, was chosen as a control (FIG. 7A).

Plots of the normalized conductance versus time recorded from Abl modified SiNW devices (FIG. 7B) exhibited reversible decreases in conductance due to competitive inhibition of ATP binding by small molecules. These data were recorded from Abl-modified SiNW devices using solutions containing 100 nM ATP and 50 nM small molecule, for Gleevec, 81, 82, 83, and biotin 84. The ATP and small molecules were dissolved in the same buffer as described in Example 1.

These data are displayed as normalized conductance, to compare devices with different absolute responses. Notably, the conductance decreased at constant small molecule concentration, which is indicative of the degree of inhibition, depending strongly on molecular similarity with Gleevec (Gleevec>81>82>83); the control biotin (84) showed essentially no change above background. The ordering for Gleevec, 81 and 83 was in agreement with reported inhibition constants of 25 nM, 1.5 micromolar and 9 micromolar, respectively. Molecule 82, whose K_{I} value was not found in published literature, showed clear inhibition, with a magnitude less than 81 but greater than 83.

To further characterize the small molecule binding, data were recorded as a function of the concentration of small molecule in a fixed ATP concentration of 100 nM (FIG. 7C). FIG. 7C shows normalized change in conductance (delta-G or ΔG) vs. small molecule concentration in fixed 100 nM of ATP. To correct for different absolute device sensitivity the data was plotted as the normalized delta-G: (delta-G, specific concentration)/(saturation delta-G)×100%, where delta-G is the difference between the measured and baseline conductances.

The results for Gleevec, 81, 82 and 83 exhibited linear increases in the inhibition at low concentrations, followed by saturation at higher values, while biotin 84 showed almost no concentration dependence. The data for the inhibitors also shifted systematically to right (higher inhibitor concentration), which is indicative of reduced inhibition for Gleevec (Gleevec>81>82>83). From the linear region of the data the inhibition constants for 81, 82 and 83 were estimated to be about 80 nM, 110 nM, and 1 micromolar, respectively.

These studies of Abl-functionalized SiNW devices demonstrated potential for label-free, real-time highly-sensitive detection of ATP binding and small-molecule inhibition of ATP binding to the tyrosine kinases. Moreover, this work showed that the affinities of different inhibitors could be distinguished at least semi-quantitatively with respect to their ability to interfere with agonist binding. The simplicity and direct nature of this approach offers advantages compared to traditional methods involving detection of radioactive ³²P in kinetic assays, and label-free techniques based on surface plasmon resonance, which are relatively insensitive to small molecules and require each small molecule to be immobilized and tested against binding of larger proteins. This approach is also attractive from the standpoint of requiring very little protein to make active devices, which could make studies of systems produced at low expression levels possible, and can be extended to sensor arrays using large scale assembly methods, for example, for high throughput screening. These results also demonstrate that these SiNW detection methods can be used to probe small molecule mediated inhibition of protein-protein interactions, for example, for drug discovery and chemical genetics applications.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B” can refer, in one embodiment, to A only (optionally including elements other than B); in another embodiment, to B only (optionally including elements other than A); in yet another embodiment, to both A and B (optionally including other elements); etc.

As used herein in the specification and in the claims, unless clearly indicated to the contrary, “or” should be understood to have the same meaning as “and/or” as defined above. For example, when separating items in a list, “or” and “and/or” each shall be interpreted as being inclusive, i.e., the inclusion of at least one, but also including more than one, of a number or list of elements, and, optionally, additional unlisted items. In general, the term “or” as used herein shall only be interpreted as indicating exclusive alternatives (i.e. “one or the other but not both”) when preceded by terms of exclusivity, such as “only one of” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system, comprising:
a sample exposure region comprising a reaction entity associated with a nanoscale wire; and
a first species and a second species different from the first species, each within the sample exposure region, wherein each of the first and second species is able to interact with the reaction entity or to affect interaction of the reaction entity with the other species.

2. The system of claim 1, wherein the first species is able to interact with the reaction entity to produce a product, and the second species is able to interact with the reaction entity to prevent or inhibit production of the product resulting from interaction of the first species and the reaction entity.

3. The system of claim 1, wherein the first species is able to interact with the reaction entity to produce a product, and the second species is a drug candidate able to interact with the first species, the reaction entity, or both, to affect interaction of the first species and the reaction entity.

4. The system of claim 1, wherein the first species and the second species competitively bind to the reaction entity.

5. The system of claim 1, wherein the first species and the second species uncompetitively bind to the reaction entity.

6. The system of claim 1, wherein the first species and the second species noncompetitively bind to the reaction entity.

7. The system of claim 1, wherein an interaction between the reaction entity and at least one of the first species and the second species causes a detectable change in a property of the nanoscale wire.

8. The system of claim 1, wherein an interaction between the reaction entity and the first species causes a first detectable change in a property of the nanoscale wire, and an interaction between the reaction entity and the second species causes a second detectable change in a property of the nanoscale wire, the first detectable change being different from the second detectable change.

9. The system of claim 1, wherein the reaction entity comprises a binding partner of at least one of the first species and the second species.

10. The system of claim 9, wherein the binding partner is non-specific.

11. The system of claim 9, wherein the binding partner is specific.

12. The system of claim 9, wherein the binding partner comprises a biomolecular receptor.

13. The system of claim 12, wherein the biomolecular receptor includes a moiety selected from the group consisting of DNA, a fragment of DNA, an antibody, an antigen, a protein, an enzyme, and combinations thereof.

14. The system of claim 1, wherein the reaction entity includes an entity selected from the group consisting of a nucleic acid, an antibody, a sugar, a carbohydrate, a protein, and combinations thereof.

15. The system of claim 1, wherein the reaction entity comprises a protein.

16. The system of claim 1, wherein the reaction entity comprises an enzyme.

17. The system of claim 1, wherein the reaction entity comprises a catalyst.

18. The system of claim 1, wherein the reaction entity comprises a polymer.

19. The system of claim 1, wherein the reaction entity is fastened to the nanoscale wire.

20. The system of claim 1, wherein the reaction entity is positioned within 100 nanometers of the nanoscale wire.

21. The system of claim 1, wherein the reaction entity is positioned within 50 nanometers of a nanoscale wire.

22. The system of claim 1, wherein the reaction entity is positioned within 10 nanometers of a nanoscale wire.

23. The system of claim 1, wherein the reaction entity is positioned within 5 nanometers of the nanoscale wire.

24. The system of claim 1, wherein the reaction entity is positioned within 3 nanometers of the nanoscale wire.

25. The system of claim 1, wherein the reaction entity is positioned within 1 nanometer of the nanoscale wire.

26. The system of claim 1, wherein the reaction entity is attached to the nanoscale wire through a linker.

27. The system of claim 1, wherein the reaction entity is directly attached to the nanoscale wire.

28. The system of claim 1, the reaction entity being positioned relative to the nanoscale wire such that it is electrically coupled to the nanoscale wire, wherein a detectable interaction between the reaction entity and at least one of the first and second species causes a detectable change in an electrical property of the nanoscale wire.

29. The system of claim 1, wherein the nanoscale wire comprises a semiconductor.

30. The system of claim 29, wherein the semiconductor nanoscale wire comprises silicon.

31. The system of claim 1, wherein the nanoscale wire is a nanotube.

32. The system of claim 31, wherein the nanotube includes a carbon nanotube.

33. The system of claim 1, wherein the nanoscale wire is a nanowire.

34. The system of claim 1, wherein the nanoscale wire is unmodified.

35. The system of claim 1, wherein the nanoscale wire is positioned on the surface of a substrate.

36. The system of claim 1, constructed and arranged to receive a fluidic sample in the sample exposure region.

37. The system of claim 36, wherein the sample is a gas stream.

38. The system of claim 36, wherein the sample is a liquid.

39. The system of claim 1, further comprising a detector constructed and arranged to determine a property associated with the nanoscale wire.

40. The system of claim 39, wherein the property is an electrical property.

41. The system of claim 39, wherein the property is an electromagnetic property.

42. The system of claim 39, where the property is a light emission property.

43. The system of claim 1, wherein the sample exposure region comprises a microchannel.

44. The system of claim 1, wherein the nanoscale wire is one of plurality of nanoscale wires, each of the plurality of nanoscale wires being doped with different concentrations of a dopant.

45. The system of claim 1, wherein the nanoscale wire is one of a plurality of nanoscale wires comprising a sensor.

46. The system of claim 45, wherein the plurality of nanoscale wires comprises at least 10 nanoscale wires.

47. The system of claim 45, wherein the plurality of nanoscale wires are arranged in parallel and addressed by a single pair of the electrodes.

48. The system of claim 45, wherein the plurality of nanoscale wires are arranged in parallel to each other and addressed individually by multiple pairs of electrodes.

49. The system of claim 45, wherein the plurality of nanoscale wires are different, each capable of detecting a different analyte.

50. The system of claim 45, wherein the plurality of nanoscale wires are oriented randomly.

51. A method, comprising an act of:
exposing a reaction entity associated with a nanoscale wire to a sample containing a first species and containing or suspected of containing a second species different from the first species, each species able to interact with the reaction entity and/or able to affect the interaction of the other species with the reaction entity.

52. The method of claim 51, wherein the first species is able to interact with the reaction entity to produce a product, and the second species is able to interact with the reaction entity to prevent or inhibit production of the product resulting from interaction of the first species and the reaction entity, and based upon determination of production of the product, determining the second species in the sample.

53. The method of claim 51, wherein the first species and the second species can competitively bind to the reaction entity.

54. The method of claim 51, wherein the first species and the second species can uncompetitively bind to the reaction entity.

55. The method of claim 51, wherein the first species and the second species can noncompetitively bind to the reaction entity.

56. The method of claim 51, wherein the first species is able to interact with the reaction entity, and the sample is known to contain the second species and the second species is a drug candidate suspected of having the ability to affect the interaction of the first species and the reaction entity, the method comprising determining the ability of the second species to affect the interaction.

57. The method of claim 51, further comprising determining a property associated with the nanoscale wire.

58. The method of claim 57, wherein the property is an electrical property.

59. The method of claim 51, wherein the nanoscale wire comprises a semiconductor.

60. The method of claim 59, wherein the semiconductor nanoscale wire comprises silicon.

61. The method of claim 51, wherein the nanoscale wire is a nanotube.

62. The method of claim 61, wherein the nanotube includes a carbon nanotube.

63. The method of claim 51, wherein the nanoscale wire is a nanowire.

64. The method of claim 51, wherein the reaction entity comprises a binding partner of at least one of the first species and the second species.

65. The method of claim 64, wherein the binding partner is non-specific.

66. The method of claim 64, wherein the binding partner is specific.

67. The method of claim 51, wherein the reaction entity comprises a protein.

68. The method of claim 51, wherein the reaction entity comprises an enzyme.

69. The method of claim 51, wherein the reaction entity comprises a catalyst.

70. The method of claim 51, wherein the reaction entity comprises a polymer.

71. The method of claim 51, wherein the reaction entity is fastened to the nanoscale wire.

72. The method of claim 51, wherein the reaction entity is positioned within 5 nanometers of the nanoscale wire.

73. The method of claim 51, wherein the reaction entity is attached to the nanoscale wire through a linker.

74. The method of claim 51, the reaction entity being positioned relative to the nanoscale wire such that it is electrically coupled to the nanoscale wire.

75. A method, comprising acts of:
exposing a nanoscale wire to an analyte; and
determining a binding constant between the analyte and the nanoscale wire.
